(19) United States
(12) Patent Application Publication
(10) Pub. No.: US 2025/0161331 A1
(43) Pub. Date: May 22, 2025

(54) ANTI-INFLAMMATORY DRUG-CANNABINOID-COMPRISING NANOEMULSIONS AND METHODS OF USING THE SAME

(71) Applicant: SGN Nanopharma Inc., Tampa, FL (US)

(72) Inventor: Pranav M. PATEL, Land O Lakes, FL (US)

(73) Assignee: SGN Nanopharma Inc., Tampa, FL (US)

(21) Appl. No.: 18/840,370

(22) PCT Filed: Feb. 23, 2023
(86) PCT No.: PCT/US2023/063142

§ 371 Date: Aug. 21, 2024

Related U.S. Application Data
(60) Provisional application No. 63/313,854, filed on Feb. 25, 2022.

(51) Int. Cl. A61K 31/00; A61K 9/107; A61K 31/196; A61K 31/519; A61K 31/573; A61K 47/10; A61K 47/14; A61K 47/22; A61K 47/26; A61K 47/44

(52) U.S. Cl. CPC A61K 31/658; A61K 9/1075; A61K 31/196; A61K 31/519; A61K 31/573; A61K 47/10; A61K 47/14; A61K 47/22; A61K 47/26; A61K 47/44

(57) ABSTRACT

The disclosed formulations include Diclofenac Sodium Salt and at least one cannabinoid. Other formulations include other anti-inflammatory drugs such as methotrexate and prednisone, for example. In addition, methods of using formulations to treat diseases or disorders such as arthritis, pain, inflammation and fever are disclosed.

### FIELD OF THE INVENTION

The present disclosure relates to the field of nanoemulsion formulations and methods of using nanoemulsion formulations to treat diseases or disorders, e.g., joint pain disorders such as arthritis, pain, inflammation, and fever. In particular, the present disclosure relates to formulations comprising Diclofenac Sodium Salt and at least one cannabinoid, as well as methods of using such formulations to treat diseases or disorders such as arthritis, pain, inflammation, and fever. Formulations comprising other anti-inflammatory drugs may be utilized.

### BACKGROUND

The following discussion is merely provided to aid the reader in understanding the disclosure and is not admitted to describing or constitute prior art thereto.

### I. Diclofenac Sodium Salt (DSS)

Diclofenac Sodium (DSS) is a sodium salt (C14H10Cl2NNaO2) and has a molecular weight of 318.1 g/mol. It is the sodium salt form of diclofenac, a benzene acetic acid derivate and is a nonsteroidal anti-inflammatory drug (NSAID) with analgesic, antipyretic and anti-inflammatory activity. Diclofenac sodium is a non-selective reversible and competitive inhibitor of cyclooxygenase (COX), subsequently blocking the conversion of arachidonic acid into prostaglandin precursors. This leads to an inhibition of the formation of prostaglandins that are involved in pain, inflammation, and fever. Diclofenac Sodium is often used in combination with misoprostol to prevent NSAID-induced gastric ulcers. See, e.g., “Diclofenac epolamine Monograph for Professionals”. Drugs.com. AHFS. Retrieved 22 Dec. 2018.

Diclofenac was first approved by the FDA in July 1988 under the trade name Voltaren, marketed by Novartis (previously Ciba-Geigy). Diclofenac sodium has also been used to investigate its radioprotective potential, to determine the basic viscosity and hydrodynamic values of the solubilizers and their micellar adducts, as a standard for potentiometric and fluorimetric determination of diclofenac in a sequential injection analysis system, to inhibit phase II drug metabolizing enzyme (DME) in a study to investigate the inhibitory effects of an ethanol extract of Descurainia sophia seeds on Phase I and II DMEs.

### II. Methotrexate

Methotrexate (MTX), formerly known as amethopterin, is a chemotherapy agent and immune-system suppressant. It is also known as disease-modifying antirheumatic drug (DMARD). It is used to treat cancer, autoimmune diseases, and ectopic pregnancy and for used for medical abortions. Methotrexate is also used to treat severe psoriasis, psoriatic arthritis (PsA), and rheumatoid arthritis (RA) and Juvenile Idiopathic Arthritis (JIA) in adults. MTX can be taken as a tablet, liquid, or injection. In 1985, scientists discovered that methotrexate can used to ease the symptoms such as joint pain, fatigue, redness and swelling. The common side effects of the drug may include dizziness, drowsiness, headaches, swollen or tender gums, hair loss, decreased appetite, mouth sores, rash, and diarrhea. Three years later after this discovery, methotrexate secured FDA approval for the treatment of RA and other forms of inflammatory arthritis as well. However, methotrexate can harm the liver and enables making the patient taking the drug more susceptible to get infections.

### III. Cannabinoids

A cannabinoid is one of a class of diverse chemical compounds that acts on cannabinoid receptors in cells and may include, but is not limited to, endocannabinoids (produced naturally in the body by animals, e.g., humans), the phytocannabinoids (found in cannabis and some other plants), and synthetic cannabinoids. Tetrahydrocannabinol (THC) and cannabidiol (CBD) are two of the most well-studied phytocannabinoids, as they are major constituents of the cannabis plant. There are at least 113 different cannabinoids that have been isolated from cannabis, and these cannabinoids have exhibited varied chemical and physiological effects.

Certain cannabinoids, particularly THC and CBD, have been studied for their application in reducing pain because of their anti-inflammatory properties.

Δ-9-Tetrahydrocannabinol (THC) is the primary active ingredient of the plant Cannabis Sativa. It has been found to have numerous applications in a wide variety of studies.

Cannabidiol (CBD) is a non-psychoactive compound found in the cannabis plant. Although the exact medical implications are currently being investigated, CBD has shown promise as an analgesic, anticonvulsant, muscle relaxant, anxiolytic, antipsychotic and has shown neuroprotective, anti-inflammatory, and antioxidant activity, among other currently investigated uses. See, Laprairie R B, Bagher A M, Kelly M E, Denovan-Wright E M: Cannabidiol is a negative allosteric modulator of the cannabinoid CB1 receptor. Br J Pharmacol. 2015 Oct;172(20):4790-805; and Pertwee R G: The diverse CB1 and CB2 receptor pharmacology of three plant cannabinoids: delta9-tetrahydrocannabinol, cannabidiol and delta9-tetrahydrocannabivarin. Br J Pharmacol. 2008 Jan;153(2):199-215.

The exemplary cannabinoid can be THC or CBD, or CBD isolate, referred in the specification as “CBDi” or “Ci,” for example, or combinations thereof. Other cannabinoids known in the art can be utilized. The medical site-Medical News Today states that “CBD isolate [CBDi] is a form of CBD, or cannabidiol, which is a chemical compound present in the cannabis plant. Unlike full-spectrum CBD products, CBD isolate does not contain any THC—the psychoactive component of cannabis.” See, https://www.medicalnewstoday.com/articles/cbd-isolate#what-it-is, retrieved Aug. 12, 2020. CBD isolate (CBDi or Ci) is normally 99% pure because it has undergone various extraction and purification process. Synthetic Cannabidiol (CBD) is also 99% pure and has same physical and chemical properties as CBDi and is cannabidiol sourced from chemically synthesized material.

### IV. Nanoemulsion Formulations

Ease of active pharmaceutical ingredient delivery is a key issue facing pharmaceutical companies that develop and attempt to commercialize therapeutic products. An active pharmaceutical ingredient (API) that is readily soluble in water, for example, is not difficult to formulate into a suitable dosage form. However, formulating poorly water-soluble therapeutic drugs into suitable dosage forms poses a significant challenge. This is because the human body is a water-based system; thus, as a condition of producing therapeutic activity, a drug must dissolve following administration.

Some poorly water-soluble API are never commercialized because they cannot be effectively solubilized, and therefore fail to exhibit acceptable in vivo therapeutic activity. Alternatively, the quantity of poorly water-soluble API required to be administered to achieve an acceptable level of therapeutic activity may be too great, given the poor water solubility of the agent, and result in unacceptable toxicity. Even if an API is formulated into a liquid, where the API is solubilized in a solvent, such dosage forms sometimes perform sub-optimally. For example, such dosage forms may have unpredictable properties or induce undesirable side effects. Diclofenac sodium, like many active pharmaceutical ingredients, is notoriously difficult to formulate, and even currently available ophthalmic formulations are associated with a number of side effects like pain and irritation, and the onset of therapeutic activity is slow, which may be due to the low concentration of Diclofenac sodium in the formulation.

Nanoemulsions are a colloidal particulate system in the submicron size range acting as carriers of drug molecules. The size of the particle/globules in a nanoemulsion varies from about 10 to 1,000 nm. These carriers are solid spheres, and their surface is amorphous and lipophilic.

Conventional wet milling techniques for making nanoemulsions produce a “bi-phasic” system in which the stabilized API nanoparticles are suspended in a liquid or aqueous media. However, wet milling of API has drawbacks, principally being the cost of the process. The added cost for formulating a poorly water-soluble API into a nanoparticulate composition utilizing wet milling can be prohibitive. Additionally, wet milling techniques are not well suited for processing amorphous or semi-amorphous APIs.

### V. Treatments for Joint Stiffness

Dry Joint stiffness is the feeling that the motion of a joint is limited or difficult. The feeling is not caused by weakness or reluctance to move the joint due to pain. Some people with stiffness can move the joint through its full range of motion, but this movement can require force.

While a variety of treatment options (e.g., Diclofenac sodium oral 100 mg tablet for pain management, or application of Topical Nonsteroidal anti-inflammatory drugs (NSAID) for arthritis pain such as 1% Diclofenac sodium topical gel (VOLTAREN®) exist, the available treatments possess numerous drawbacks. For instance, currently available Diclofenac oral formulations may upset stomach, nausea, heartburn, diarrhea, constipation, gas, headache, drowsiness, and dizziness, hearing changes (such as ringing in the ears), mental/mood changes, difficult/painful swallowing, symptoms of heart failure (such as swelling ankles/feet, unusual tiredness, unusual/sudden weight gain). The side effects included with usage of topical Diclofenac gel is irritation and rash on the site of application. Moreover, once a patient begins an arthritis treatment regimen that includes application of currently available Diclofenac sodium formulations, it may take two to seven days or more before the patient achieves any sign of clinical improvement. The other major challenge is to limit the penetration of Diclofenac sodium formulations in the blood stream or otherwise increasing the blood pressure may occur.

There are only two FDA approved drugs containing cannabinoids; Marinol and Epidiolex, discussed as follows.

Marinol is an oral medication used by adults to treat loss of appetite associated with AIDS and nausea/vomiting associated with cancer treatment (chemotherapy). The active ingredient Dronabinol I in Marinol is a man-made form of cannabis. The recommended dosage varies per indication; for anorexia, it is 2.5 mg used twice daily before lunch and dinner. For symptoms associated with chemotherapy, the dosage is 5 mg used 1-3 hours before the start of treatment, then every 2 to 4 hours after treatment. The biggest setback of the drug is dronabinol's greasy consistency which makes the compound difficult and expensive to purify. Also, because dronabinol does not dissolve readily in water, only a fraction of the orally ingested compound (about 10 to 20%) reaches the patient's circulation. See, Mack A, Joy J. Marijuana as Medicine? The Science Beyond the Controversy. Washington (DC): National Academies Press (US); 2000. 10, PHARMACEUTICALS FROM MARIJUANA. Available from: https://www.ncbi.nlm.nih.gov/books/NBK224399.

Epidiolex is an oral cannabidiol solution used for the treatment of seizures associated with two rare and severe forms of epilepsy, Lennox-Gastaut syndrome and Dravet syndrome, in patients two years of age and older. It has a starting dosage of 2.5 mg/kg taken by mouth twice daily and a maintenance dosage of 5 mg/kg taken by mouth twice daily. A major setback of this drug is the stability; the shelf life is only 12 weeks.

Some challenges associated with THC included low aqueous solubility and high lipophilicity. Likewise, there problems with cannabinoids such as CBD and CBDi. While the biological activity of cannabinoids such as CBD and CBDi is attractive, the stability of these compounds has previously presented challenges. For instance, when incorporated into a solution as a tea, CBD concentrations decreased by as much as 50% when stored at room temperature for 3-7 days, and similar decreases of up to 38% were seen in cannabis oil formulations across the same timeframe (see, e.g., Pacifici et al., Clin. Chem. Lab. Med., 55(1):1555-1563 (2017)).

Therefore, there is an obvious market needs for cannabinoid-based drugs with better stability than already existing products, and well as a relatively easier production process.

There is a need in the art for improved therapies for treating joint pain conditions such as arthritis that will provide patients with an alternative that does not cause irritation and rash on the skin and has a faster therapeutic onset.

### SUMMARY OF THE INVENTION

In one example, a formulation comprises one or more anti-inflammatory drugs. In yet another example, the formulation comprises one or more anti-inflammatory drugs in combination with one or more cannabinoids. The anti-inflammatory drug may be a non-steroidal anti-inflammatory drug, for example.

In one example, a formulation comprises Diclofenac sodium or a salt (e.g., Diclofenac Sodium Salt (DSS) or derivative thereof and at least one cannabinoid or a salt or derivative thereof suspended in an emulsion. In yet another example, the formulation comprises diclofenac sodium or its salt alone; (2) a water concentration range of about 35-38%; (3) an oil concentration range of about 35 to 38%; and (4) a pH range of 7.0 to 8.3.

In one example, the formulation comprises (a) an aqueous phase; and (b) a pharmaceutically acceptable oil. One example of a pharmaceutically acceptable oil is an oil having at least one oxidizable metal of less than one part per million in said oil, wherein the at least one oxidizable metal is one or more of the following: a copper content of less than one part per million in said oil; a chromium content of less than one part per million in said oil; a lithium content of less than one part per million in said oil; and a molybdenum content of less than one part per million in said oil; and at least one cannabinoid or a salt or derivative thereof dissolved in the pharmaceutically acceptable oil; the at least one cannabinoid or a salt or a derivative thereof having a weight percentage of up to 75% of the formulation. An exemplary weight percentage range of the at least one cannabinoid or a salt or a derivative is from 0.01% weight/by weight to 75% weight/weight of the formulation. The term “weight percentage” herein as used in the claims and the specification is intended to mean “weight/weight of the formulation.” For example, if a cannabinoid is 75% of the formulation, this means 750 milligrams of cannabinoid per 1 gram of the formulation. The oil content is in the range of about 0.01% w/w to about 99% w/w of the at least one pharmaceutically acceptable oil having at least one oxidizable metal of less than one part per million in said oil.

However, the pharmaceutically acceptable oil used in the formulation comprising Diclofenac sodium or a salt or derivative thereof and at least one cannabinoid or a salt or derivative thereof suspended in an emulsion need not be the mentioned oil having the described metal contents. Other pharmaceutically acceptable oils may be used.

In yet another example, the formulation comprises 0.1% DSS and CBDi and THC. This formulation may have a range of 4-10% oil, 0.2 to 0.3% ethanol, 90-95% water and a pH range of 7.0 to 8.

In another example, the formulation comprises 1% DSS and CBDi. This formulation may have an oil percentage range of 33-40%, a water concentration range from 37.2 to 47% and a pH range of 7.8-8.4.

In yet another example, the formulation may be a combination of methotrexate, DSS and CBDi. The formulation may have a range of 0.5 to 1% methotrexate, 1 to 5% DSS, 1-10% CBDi, a water concentration of 35-42%, an oil concentration range of 37-63% and a pH range of 7.0 to 8.2.

In one example, a formulation comprises prednisone or a salt or derivative thereof and at least one cannabinoid or a salt or derivative thereof suspended in an emulsion. In yet another example, the formulation comprises prednisone alone; (2) a water concentration range of about 35-38%; (3) an oil concentration range of about 35 to 38%; and (4) a pH range of 7.0 to 8.3. Other water and concentration ranges are contemplated. Other pH ranges are contemplated.

In yet another example, the formulation comprises 0.1% prednisone and CBDi and THC. This formulation may have a range of 4-10% oil, 0.2 to 0.3% ethanol, 90-95% water and a pH range of 7.0 to 8. This application may be for ophthalmic usages. Other concentrations of oil, ethanol, water and pH ranges are contemplated.

In yet another example, the formulation comprises 1% prednisone and CBDi. This formulation may have an oil percentage range of 33-40%, a water concentration range from 37.2 to 47% and a pH range of 7.8-8.4. Other concentrations of oil, water and pH ranges are contemplated. In this example, transdermal applications are contemplated.

Regarding the foregoing formulations of prednisone, those formulations may include one or more other non-steroidal anti-inflammatory drugs such as Diclofenac sodium or a salt (e.g., Diclofenac Sodium Salt (DSS) or derivative thereof or methotrexate.

Excipients are traditionally considered as pharmacologically inert chemicals that are used in various kinds of formulations. Excipients used in formulations can also actively participate in drug-excipient interactions that can eventually compromise the stability of the product and therapeutic role of the drug component(s) following drug decomposition. However, the magnitude of decomposition of the drug substance(s) highly depends on the chemical configuration, quantity of the drug, and physical and chemical properties of excipients used in the formulations. One of the major factors of oxidative degradation of drugs are metal ions present in excipients. To avoid metal induced oxidative decomposition of pharmaceutical drugs, it is especially important to select appropriate excipients.

Pharmaceutically acceptable oils are used in the exemplary formulation comprising Diclofenac sodium or a salt or derivative thereof and at least one cannabinoid or a salt or derivative thereof suspended in an emulsion. In one example of a pharmaceutically acceptable oil, a pharmaceutically acceptable oil has at least one oxidizable metal of less than one part per million in said oil, wherein the at least one oxidizable metal is one or more of the following: a copper content of less than one part per million in said oil; a chromium content of less than one part per million in said oil; a lithium content of less than one part per million in said oil; and a molybdenum content of less than one part per million in said oil. Such exemplary oils include Brand A or oils, such as include Medium Chain Triglycerides Oils, Brand A olive oil, or Brand A soybean oil or any other pharmaceutically acceptable oil having the mentioned metal contents.

However, the pharmaceutically acceptable oil need not be the foregoing mentioned oil having the mentioned metal contents and include other oils such as Brand B oils such as Brand B sesame oil or Brand B olive oil, for example, Brand C oils and Brand D oils.

Such exemplary formulations can be for oral or topical use, and combinations thereof. Any nonsteroidal anti-inflammatory drug known in the art can be used in combination with least one cannabinoid having a weight percentage of at least 0.25%, whether alone or in combination with other nonsteroidal anti-inflammatory drug in the mentioned pharmaceutically acceptable oils described above. In the foregoing examples or any of the later examples described in the specification, CBDi can be alternatively used instead of CBD. For example, this CBDi replacement occurs for combinations with THC or in combinations with THC and DSS (Diclofenac Sodium Salt) or with DSS (Diclofenac Sodium Salt) alone.

The present disclosure provides novel nanoemulsion formulations comprising at least one cannabinoid or a salt or derivative thereof and, in some embodiments, Diclofenac Sodium or a salt or derivative thereof.

Methods of treatment or foreseeable uses of the disclosed nanoemulsion formulations include treating arthritic conditions such as joint pain as well as applications as NSAID for pain management conditions. Combining (1) cannabinoids or a salt or derivative thereof and (2) Diclofenac Sodium or a salt or derivative thereof into a formulation for treating arthritic conditions such as joint pain provides numerous benefits, including but not limited to, (i) decreasing pain, less irritation, and other adverse events associated with administration of diclofenac sodium alone; (ii) decreasing the amount of time needed for therapeutic onset as compared to application of Diclofenac sodium alone; and (iii) allowing for higher doses of Diclofenac Sodium to be safely administered. Additionally, the disclosed formulations comprising at least one cannabinoid or a salt or derivative thereof (e.g., THC or CBD or CBDi), either alone or in combination with Diclofenac sodium or a salt or derivative thereof, provide anti-inflammatory and analgesic effects when applied on the knee of a patient in need. CBD isolate can be replaced with synthetic CBD as well in any of these nanoformulations.

Accordingly, in one aspect, the present disclosure provides nanoemulsion formulations comprising at least one cannabinoid or a salt or derivative thereof formulated in an emulsion, the emulsion comprising an aqueous phase, at least one pharmaceutically acceptable oil, and at least one surfactant. The formulation can further comprise Diclofenac sodium or a salt or derivative thereof.

In some embodiments, the formulation may further comprise at least one organic solvent. In some embodiments, the organic solvent may be an alcohol, such as for example, ethanol, methanol, isopropyl alcohol, glycerol, derivatives thereof, and any combination thereof.

In another aspect, the present disclosure provides nanoemulsion formulations comprising droplets having an average particle size of less than about 1 micron and (i) about 0.5 to about 2% (w/w) Diclofenac sodium or a derivative or salt thereof; (ii) about 1 to about 5% (w/w) of at least one cannabinoid or a derivative or salt thereof; (iii) about 10 to about 90% (w/w) water; (iv) about 10 to about 60% (w/w) of at least one pharmaceutically acceptable oil; (v) about 1 to about 15% of at least one surfactant; and (vi) about 0.05 about 0.5% (w/w) of at least one organic solvent.

In some embodiments, the formulation may comprise droplets having an average particle size of less than about 1 micron and (i) about 0.01 to about 2% (w/w) Diclofenac sodium or a derivative or salt thereof; (ii) about 0.01 to about 0.5% (w/w) of at least one cannabinoid or a derivative or salt thereof; (iii) about 10 to about 90% (w/w) water; (iv) about 10 to about 60% (w/w) of at least one pharmaceutically acceptable oil; (v) about 1 to about 15% (w/w) of at least one surfactant; and (vi) about 0.05 about 0.5% (w/w) of at least one organic solvent.

Another embodiment is directed to a nanoemulsion formulation comprising droplets having an average particle size of less than about 1 micron and: (i) about 0.01 to about 0.5% (w/w) tetrahydrocannabinol (THC) or a salt or derivative thereof; (ii) about 0.01 to about 15% (w/w) cannabidiol (CBD) or CBDi or a salt or derivative thereof; (iii) about 10 to about 90% (w/w) water; (iv) about 10 to about 60% (w/w) of at least one pharmaceutically acceptable oil; (v) about 1 to about 15% (w/w) of at least one surfactant; and (vi) about 0.05 to about 0.5% (w/w) of at least one organic solvent. The formulation can further comprise Diclofenac sodium or a salt or derivative thereof.

In some embodiments, the emulsion droplet size may be selected from the group consisting of less than about 1 micron, less than about 900 nm, less than about 800 nm, less than about 700 nm, less than about 600 nm, less than about 500 nm, less than about 450 nm, less than about 400 nm, less than about 350 nm, less than about 300 nm, less than about 250 nm, less than about 200 nm, less than about 150 nm, and less than about 100 nm.

In some embodiments, the Diclofenac sodium or a salt or derivative thereof can form particles that are suspended in the nanoemulsion, and these particles may have an average particle size selected from the group consisting of less than about 1 micron, less than about 900 nm, less than about 800 nm, less than about 700 nm, less than about 600 nm, less than about 500 nm, less than about 450 nm, less than about 400 nm, less than about 350 nm, less than about 300 nm, less than about 250 nm, less than about 200 nm, less than about 150 nm, and less than about 100 nm.

In some embodiments, the Diclofenac sodium or a salt or derivative is present at a therapeutically effective amount, which can be at a concentration of about 1% to about 2.0%, 0.25% to about 0.05% (w/w), or 0.01 to about 0.5% (w/w), 0.025% to 2.0% (w/w), or any amount in-between these values.

In some embodiments, the cannabinoid may be present at a concentration of about 1% to about 5% (w/w), 0.01 to about 10.0% (w/w), or any amount in-between these values. In some embodiments, the at least one cannabinoid or a salt or derivative thereof may be cannabidiol (CBD), CBDi.

In some embodiments, the pharmaceutically acceptable oil is selected from the group consisting of almond oil, apricot seed oil, borage oil, canola oil, coconut oil, corn oil, cotton seed oil, fish oil, jojoba bean oil, lard oil, linseed oil (boiled), Macadamia nut oil, mineral oil, olive oil, peanut oil, Brand A Safflower oil, sesame oil, soybean oil, squalene, sunflower seed oil, tricaprylin (1,2,3-trioctanoyl glycerol), and wheat germ oil. In some embodiments, the pharmaceutically acceptable oil may comprise a medium chain triglyceride (e.g., BRAND A MEDIUM CHAIN TRIGLYCERIDES).

In other embodiments, the pharmaceutically acceptable oil is an oil having at least one oxidizable metal of less than one part per million in said oil, wherein the at least one oxidizable metal is one or more of the following: a copper content of less than one part per million in said oil; a chromium content of less than one part per million in said oil; a lithium content of less than one part per million in said oil; and a molybdenum content of less than one part per million in said oil. Such oils include BRAND A MEDIUM CHAIN TRIGLYCERIDES or oils made by Brand A Inc., such as include Brand A Sesame oil, Brand A olive oil, Brand A Safflower oil or Brand A soybean oil.

In some embodiments, the surfactant may be a suitable ionic or non-ionic surfactant, such as a polysorbate or polyethylene glycol. In some embodiments, the polysorbate may be polysorbate 80. In some embodiments, two or more surfactants can be used. In one such example, polysorbate 20 and polysorbate 80 are used.

In some embodiments, the formulation may be stable at room temperature for a period of time selected from the group consisting of at least about 3 months, at least about 4 months, at least about 5 months, at least about 6 months, at least about 7 months, at least about 8 months, about at least about 9 months, at least about 10 months, at least about 11 months, at least about 12 months, at least about 13 months, at least about 14 months, at least about 15 months, at least about 16 months, at least about 17 month, and at least about 18 months.

In some embodiments, the formulation may be multiphasic or triphasic. For instance, in some embodiments, the Diclofenac sodium or a salt or derivative thereof and/or at least one cannabinoid or a salt or derivative thereof may be present as (i) a suspension of nanoparticles in the oil phase; and (ii) dissolved in the aqueous phase, while in some embodiments, the Diclofenac sodium and/or at least one cannabinoid may be present as (i) a suspension, (ii) dissolved in the oil phase, and (iii) dissolved in the aqueous phase.

In some embodiments, the at least one cannabinoid, such as CBD, CBDi or a combination thereof, are present (i) as a suspension of nanoparticles in the oil phase, and (ii) dissolved in the hydrophilic phase.

In some embodiments of the foregoing aspects, the formulations described herein are dermal formulations.

In another aspect, the present disclosure provides methods of treating arthritic conditions, including but not limited to joint pain, comprising administering to the skin of a subject in need a nanoemulsion formulation according to any one of the foregoing aspects or embodiments. A “subject in need” in one example, can be a subject with a dermal condition.

In some embodiments, a formulation comprising a combination of Diclofenac sodium or a salt or derivative thereof, combined with at least one cannabinoid or a salt or derivative thereof, creates or results in less skin irritation when administered to the skin of an individual as compared to a Diclofenac sodium formulation that does not comprise at least one cannabinoid. This difference in skin irritation, can be measured by, for example, an acute eye irritation/corrosion test in an animal.

In some embodiments, a formulation comprising a combination of Diclofenac sodium or a salt or derivative thereof, combined with at least one cannabinoid or a salt or derivative thereof, may have a faster onset of therapeutic activity when applied to the skin of an individual as compared to a Diclofenac sodium formulation that does not comprise at least one cannabinoid. This difference in onset of therapeutic activity can be determined by, for example, as follows. For instance, in some embodiments, the onset of therapeutic activity occurs in a period of time selected from the group consisting of less than about 1 months, less than about 15 days, less than about 10 days, less than about 7 days, less than about 5 days, and less than about 1 day.

The foregoing general description and following detailed description are exemplary and explanatory and are intended to provide further explanation of the invention as claimed. Other objects, advantages, and novel features will be readily apparent to those skilled in the art from the following brief description of the drawings and detailed description of the invention.

### DETAILED DESCRIPTION

The examples and drawings provided in the detailed description are merely examples and should not be used to limit the scope of the claims in any claim construction or interpretation.

The present disclosure provides novel nanoemulsion formulations comprising at least one cannabinoid or a salt or derivative thereof (e.g., CBD or CBDi), and, in some embodiments, an additional pharmaceutically active compound, such as Diclofenac sodium or a salt or derivative thereof. The present disclosure also provides uses and methods or treatment of arthritic conditions, such as joint pain, utilizing the disclosed nanoemulsion formulations.

The present invention provides composition and methods for treating arthritic conditions, such as but not limited to joint pain. The present invention provides dermal applications for anti-inflammatory conditions, for example.

The disclosed formulations are unique in that the active compounds (e.g., Diclofenac sodium, CBD, CBDi or salts or derivatives thereof) are multiphasic as a result of preparation using high pressure (i.e., >10,000 psi) homogenization. In other words, the active agents may be present in three phases of the nanoemulsion: (1) dissolved in the aqueous phase, (2) dissolved in the oil/lipophilic phase, and (3) in a particulate form in suspension in the aqueous and/or oil phases.

The disclosed formulations are exceptionally stable. For example, the compositions may be stable at room temperature for more than 12 months, whereas cannabinoids generally tend to become unstable and decompose within a matter of days after being placed in solution, even when held at 4° C.

From a clinical perspective, the disclosed formulations represent a significant improvement over prior topical dermal treatments for conditions like arthritis. This is because the formulations described herein produce a faster onset of therapeutic activity and reduced irritation as compared to conventional products that include Diclofenac sodium as the only pharmaceutically active compound (e.g., Voltarin®).

Accordingly, the disclosed nanoemulsion formulations and uses/methods of treatment of ophthalmic conditions with the disclosed formulations provide a significant, unexpected benefit in the art.

### Nanoemulsion Formulations

### 1. Diclofenac Sodium

In one embodiment of the invention, the nanoemulsions described herein comprise Diclofenac sodium or a salt or derivative thereof. Diclofenac sodium has traditionally been used as an immunosuppressant in patients that have received organ transplants. Arthritis formulations of Diclofenac sodium (e.g., VOLTERIN®) have also proven to be beneficial in treating joint pain. However, when used to treat joint pain, Diclofenac sodium monotherapy has at least two clinical drawbacks. First, Diclofenac sodium is known to cause irritation or redness when applied to the skin. This irritation and pain usually does not subside over time and can threaten or completely undermine patient compliance. Second, because Diclofenac sodium delivery using conventional arthritis formulations is inefficient, it may take more than three to six months of treatment before a given patient begins seeing a clinical improvement in the management of their joint pains.

### 2. Cannabinoids

The nanoemulsions of the invention can comprise one or more cannabinoids, or a salt or derivative thereof. Cannabinoids are a class of biologically active molecules that can bind to and agonize the cannabinoid receptors CB1 and CB2. Cannabinoids may be isolated from the cannabis plant (e.g., CBD and CBDi), synthetically derived, or occur endogenously in mammals (e.g., anandamide, 2-AG, noladin ether, virodhamine, and N-arachidonyl dopamine (NADA)).

Cannabidiol (CBD), which is shown in Formula 1 below, is among the most active cannabinoids that can be isolated from the cannabis plant. This compound possesses anti-inflammatory properties and analgesic properties.

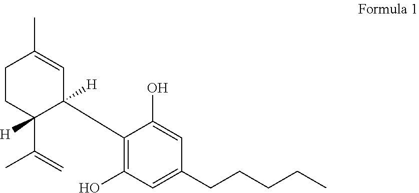 [SMILES: C=C(C)[C@@H]1CCC(C)=C[C@H]1c1c(O)cc(CCCCC)cc1O]

Cannabinoids, in general, CBD or CBDi, may be incorporated into the disclosed nanoemulsions for the treatment of arthritis conditions such as joint pain. Joint pain is associated with pain, inflammation, irritation of the joints, that can be directly addressed by the anti-inflammatory and analgesic properties of cannabinoids such as CBD or CBDi.

As mentioned in the background, stability of CBD and CBDi remains a problem. In contrast, the cannabinoid nanoemulsion formulations disclosed herein are stable for at least 3 months at room temperature and may be stable for more than about 4 months, more than about 5 months, more than about 6 months, more than about 7 months, more than about 8 months, more than about 9, months, more than about 10 months, more than about 11 months, or more than about a year.

It is believed that the addition of cannabinoids to DSS Nanoformulations can help in the treatment of joint pain that results by arthritis. THC and CBD exert their effect by interacting with the body's endocannabinoid system. This system is made up on a network of receptors; two of these have been identified as CB1. (Cannabinoid 1) an CB2 (Cannabinoid 2). CB2 receptors have been found to be present and functionally active in the bones. See, Dunn S L, Wilkinson J M, Crawford A, Bunning R A D, Le Maitre C L (2016) Expression of cannabinoid receptors in human osteoarthritic cartilage: implications for future therapies, Cannabis and Cannabinoid Research 1:1, 3-15, DOI: 10.1089/can.2015.0001

CB2 receptors are present in the joints. See, La Porta C, Bura S A, Aracil-Fernández A, Manzanares J, Maldonado R. Role of CB1 and CB2 cannabinoid receptors in the development of joint pain induced by monosodium iodoacetate. Pain. 2013 Jan;154(1):160-174. doi: 10.1016/j.pain.2012.10.009. Epub 2012 Oct. 23. PMID: 23199705.

Although the mode of action of THC hasn't been fully understood, it is believed to be a partial agonist of both CB1 and CB2 receptors present in the bones and might be involved in bone stiffness reducer. See, He and F, Song Z H. Molecular and cellular changes induced by the activation of CB2 cannabinoid receptors in trabecular meshwork cells. Mol Vis. 2007;13:1348-56 and Njie Y F, Qiao Z, Xiao Z, Wang W, Song Z H. Narachidonylethanolamide-induced increase in aqueous humor outflow facility. Invest Ophthalmol Vis Sci. 2008;49:4528-34.

Any suitable amount of cannabinoid can be used in the disclosed nanoemulsion formulations. For example, the nanoemulsion formulation can comprise between about 0.01% and about 10% of at least one (i.e., one or two or three or more) cannabinoid. For example, in formulations containing CBD or CBDi, the formulation may comprise between about 0.01% and about 5% (w/w) CBD. Similarly, in formulations comprising only one of either CBD or CBDi, the formulation may comprise between about 0.01% and about 10% (w/w) CBD or CBDi. Thus, in some embodiments the disclosed formulations may comprise about 0.01, about 0.02, about 0.03, about 0.04, about 0.05, about 0.06, about 0.07, about 0.08, about 0.09, about 0.1, about 0.2, about 0.3, about 0.4, about 0.5, about 0.6, about 0.7, about 0.8, about 0.9, about 1.0, about 1.05 about 1.1, about 1.15, about 1.2, about 1.25, about 1.3, about 1.35, about 1.4, about 1.45, about 1.5, about 1.55, about 1.6, about 1.65, about 1.7, about 1.75, about 1.8, about 1.85, about 1.9, about 1.95, about 2.0, about 2.05 about 2.1, about 2.15, about 2.2, about 2.25, about 2.3, about 2.35, about 2.4, about 2.45, about 2.5, about 2.55, about 2.6, about 2.65, about 2.7, about 2.75, about 2.8, about 2.85, about 2.9, about 2.95, about 3.0, about 3.05 about 3.1, about 3.15, about 3.2, about 3.25, about 3.3, about 3.35, about 3.4, about 3.45, about 3.5, about 3.55, about 3.6, about 3.65, about 3.7, about 3.75, about 3.8, about 3.85, about 3.9, about 3.95, about 4.0, about 4.05 about 4.1, about 4.15, about 4.2, about 4.25, about 4.3, about 4.35, about 4.4, about 4.45, about 4.5, about 4.55, about 4.6, about 4.65, about 4.7, about 4.75, about 4.8, about 4.85, about 4.9, about 4.95, or about 5.0% and up to 10% (w/w) of at least one (i.e., one or two or three or more) cannabinoid (e.g., CBD or CBDi). In addition, the nanoemulsion formulations of this invention can comprise between about 0.01% and about 15%, between about 0.01% and about 20%, between about 0.01% and about 25%, between about 0.01% and about 30%, between about 0.01% and about 35%, between about 0.01% and about 40%, between about 0.01% and about 45%, or between about 0.01% and about 50% of at least one (i.e., one or two or three or more) cannabinoid, or a salt or derivative thereof.

It should further be noted that when more than one cannabinoid is present in a formulation, the cannabinoids do not need to be incorporated in identical concentrations. In other words, when THC and CBD or CBDi are both present, the concentration of THC may be higher than the concentration of CBD or CBDi and vice versa. For example, in some embodiments, CBD may be present at a concentration of about 0.1% to about 10% (w/w) and THC may be present at a concentration of about 0.01% to about 0.03% (w/w).

When incorporated into the disclosed nanoemulsion formulations, these clinical drawbacks are overcome. The analgesic properties of cannabinoids such CBD decrease the skin irritation associated with Diclofenac sodium application, and the anti-inflammatory properties of the cannabinoids decrease Diclofenac sodium-induced irritation. Moreover, the multiphasic nature of the disclosed nanoemulsions, along with the small, uniform size of the globules and particles therein, makes the disclosed formulations less irritating and more palatable for patients.

Additionally, because cannabinoids such as CBD can improve treatment of arthritis conditions such as joint pain directly and function via distinct mechanisms from Diclofenac sodium, the disclosed formulations that incorporate Diclofenac sodium will produce a clinical response much faster than an arthritis formulation that comprises Diclofenac sodium alone. Indeed, the disclosed formulations may produce a significant clinical benefit or improvement in one or more signs or symptoms of joint pain in less than about 3 months, less than about 2.5 months, less than about 2 months, less than about 1.5 months, less than about 1 month, less than about 3 weeks, less than about 2 weeks, less than about 1 week, or with 1, 2, 3, 4, 5, 6, or 7 days of commencement of treatment with the disclosed compositions.

Moreover, because the combination of Diclofenac sodium with at least one cannabinoid (e.g., CBD or CBDi) decreases negative side effects of arthritis, and side effects from using Diclofenac sodium such as and irritation and stomach ulcers, more concentration of Diclofenac sodium may be included in the disclosed formulations than what is included in conventional arthritis formulations that included Diclofenac sodium without a cannabinoid. For instance, VOLTERIN® is a 1% NSAID arthritis topical of Diclofenac sodium.

In contrast, for the purposes of the present disclosure, any suitable amount of Diclofenac sodium can be used in the disclosed nanoemulsion formulations. For example, the nanoemulsion formulation can comprise between about 1% and about 3% (w/w) Diclofenac sodium. Thus, the disclosed formulations may comprise about 1% to about 3% (w/w) Diclofenac sodium, about 1.5% to about 2.5% (w/w) Diclofenac sodium, about 2% to about 2% (w/w) Diclofenac sodium, about 2.5% to about 1.5% (w/w) Diclofenac sodium, about 3% to about 1% (w/w) Diclofenac sodium or a salt or derivative thereof, or any percent range in between any of these concentration ranges. Thus, in some embodiments the disclosed formulations may comprise about 1, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9 about 3.0% (w/w) of Diclofenac sodium or a salt or derivative thereof. The nanoemulsion formulations of this invention can also comprise between about 1% and about 4% (w/w), between about 1% and about 5% (w/w), between about 1% and about 6% (w/w), between about 1% and about 7% (w/w), between about 1% and about 8% (w/w), between about 1% and about 9% (w/w), or between about 1% and about 10% (w/w) Diclofenac sodium, or a salt or derivative thereof.

Accordingly, these weight percentages of Diclofenac sodium in the present disclosure are higher than those in current commercial formulations such as VOLTERIN®, a 1% NSAID arthritis topical of Diclofenac sodium.

It should also be noted that multiple Diclofenac sodium compounds are known in the art, including Diclofenac sodium A, B, C, D, E, H, and L. The presently disclosed nanoemulsion formulations can incorporate any of these Diclofenac sodium compounds, including salts and derivatives thereof.

Definition of “Diclofenac sodium” as used in the specification. Unless indicated to the contrary, the term “Diclofenac sodium” as used in this disclosure is understood to be Diclofenac sodium A, which is shown in Formula 2 below.

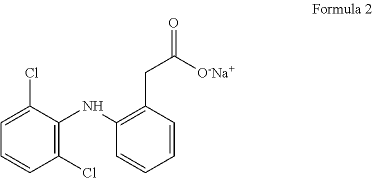

### Nanoemulsion

As described above, the compositions of the invention comprise a nanoemulsion combined with at least one cannabinoid or a salt or derivative thereof (e.g., CBD or CBDi) and, optionally, Diclofenac sodium or a salt or derivative thereof to make a nanoemulsion formulation useful for treating arthritis conditions such as joint pain. The nanoemulsion formulations may comprise an aqueous phase, an oil/lipophilic phase, at least one solvent, and/or at least one surfactant.

Nanoemulsions of the present disclosure can be produced, for example, using high pressure homogenization. High energy input, through high velocity homogenization or vigorous stirring, is a preferred process. The high energy processes reduce the size of the emulsion droplets, thereby exposing a large surface area to the surrounding aqueous environment. High shear processes are preferred, as low shear processes can result in larger particle/droplet sizes. Accordingly, in some embodiments the disclosed nanoemulsions may be produced by submitting a mixture comprising an aqueous phase, oil phase, and at least one cannabinoid or a salt or derivative thereof and, optionally, Diclofenac sodium or a salt or derivative thereof to multiple rounds (e.g., 2, 3, 4, 5, 6, 7, 8, 9, or 10 or more) of high-pressure homogenization.

High pressure homogenization includes homogenization at pressures greater than about 10,000 psi, for example, about 11,000 or more; about 12,000 or more; about 13,000 or more; about 14,000 or more; about 15,000 or more; about 16,000 or more; about 17,000 or more; about 18,000 or more; about 19,000 or more; about 20,000 or more; about 21,000 or more; about 22,000 or more; about 23,000 or more; about 24,000 or more; about 25,000 or more; about 30,000 or more; about 35,000 or more; or about 40,000 or more psi.

In some embodiments, the disclosed nanomeulsion formulations may undergo multiple rounds of extrusion or homogenization at high pressure to obtain a product with consistent, unimodal particle/globule size distribution and a desirable average globule or particle size. Thus, the formulations may undergo 1, 2, 3, 4, 5, 6, 7, 8, 9, 10, 11, 12, 13, 14, or 15 or more rounds of extrusion or homogenization at high pressure homogenization.

The disclosed high pressure/high shear processes produce nanoemulsion formulations that are uniquely multiphasic meaning the active compounds (e.g., CDB, Diclofenac sodium) may be (i) dissolved in the aqueous phase, (ii) dissolved in the oil/lipid phase, and (iii) suspended in a particulate form in the aqueous and/or oil/lipid phase. Moreover, the formulations are exceptionally stable.

“Stability” or “Stable” as defined in the specification. The terms “stability” or “stable” as defined in the present disclosure includes three definitions, as follows. For the purposes of the present disclosure, the terms “stability” or “stable” are intended to mean that the formulation does not separate or precipitate and that the globule and particle sizes do not increase more than 15% over a given period of time. Additionally, the terms “stability” or “stable” can also be indicated by the ability of the formulation to prevent loss or degradation of the active products, such as CBD, and/or Diclofenac sodium. For example, a stable nanoemulsion formulation may see a decrease of less than about 10%, less than about 9%, less than about 8%, less than about 7%, less than about 6%, less than about 5%, less than about 4%, less than about 3%, less than about 2%, or less than about 1% of an active compound after storage for a given period of time (e.g., 3-4 months) at a given temperature (e.g., room temperature).

In some embodiments, the disclosed nanoemulsion formulations can be stable at about 20° C. to about 25° C. (i.e., “room temperature”) for a time period of at least up to about 1 month, at least up to about 2 months, at least up to about 3 months, at least up to about 4 months, at least up to about 5 months, at least up to about 6 months, at least up to about 7 months, at least up to about 8 months, at least up to about 9 months, at least up to about 10 months, at least up to about 11 months, at least up to about 12 months, at least up to about 18 months, at least up to about 2 years, at least up to about 2.5 years, at least up to about 3 years, at least up to about 3.5 years, at least up to about 4 years, at least up to about 4.5 years, at least up to about 5 years, at least up to about 5.5 years, at least up to about 6 years, at least up to about 6.5 years, or at least up to about 7 years.

In some embodiments, the disclosed nanoemulsion formulations can be stable at about 4° C. for a time period of at least up to about 1 month, at least up to about 3 months, at least up to about 6 months, at least up to about 12 months, at least up to about 18 months, at least up to about 2 years, at least up to about 2.5 years, at least up to about 3 years, at least up to about 3.5 years, at least up to about 4 years, at least up to about 4.5 years, at least up to about 5 years, at least up to about 5.5 years, at least up to about 6 years, at least up to about 6.5 years, or at least up to about 7 years.

In some embodiments, the disclosed nanoemulsion formulations can be stable at about −20° C. for a time period of at least up to about 1 month, at least up to about 3 months, at least up to about 6 months, at least up to about 12 months, at least up to about 18 months, at least up to about 2 years, at least up to about 2.5 years, at least up to about 3 years, at least up to about 3.5 years, at least up to about 4 years, at least up to about 4.5 years, at least up to about 5 years, at least up to about 5.5 years, at least up to about 6 years, at least up to about 6.5 years, or at least up to about 7 years.

### Aqueous Phase

The aqueous phase can comprise any type of aqueous phase including, but not limited to, water (e.g., H₂O, distilled water, purified water, water for injection, de-ionized water, tap water) and solutions (e.g., phosphate buffered saline (PBS) solution). In certain embodiments, the aqueous phase comprises water at a pH of about 4 to 10, or about 6 to 8. For example, the pH may be about 4, about 5, about 6, about 7, about 8, about 9, or about 10. The water can be deionized (hereinafter “DiH₂O”). In some embodiments the aqueous phase comprises phosphate buffered saline (PBS). The aqueous phase may further be sterile and pyrogen free.

The aqueous phase of the formulation may make up about 10% to about 60% (w/w) of the formulation, about 20% to about 50% (w/w) of the formulation, or about 30% to about 40% (w/w) of the formulation. For example, the aqueous phase may be about 10%, about 15%, about 20%, about 25%, about 30%, about 35%, about 40%, about 45%, about 50%, about 55%, or about 60% (w/w) of the formulation.

### Oil Phase

The oil in the nanoemulsion formulations of the present disclosure can be any cosmetically or pharmaceutically acceptable oil. The oil can be volatile or non-volatile, and may be chosen from animal oil, vegetable oil, natural oil, synthetic oil, hydrocarbon oils, silicone oils, semi-synthetic derivatives thereof, and combinations thereof.

The presently disclosed nanoemulsions are not limited to particular oil. A variety of oils are contemplated, including, but not limited to, sesame, soybean, avocado, squalene, olive, canola, corn, rapeseed, Brand A Safflower, sunflower, fish, flavor, and water insoluble vitamins. Suitable oils include, but are not limited to, mineral oil, squalene oil, flavor oils, silicon oil, essential oils, water insoluble vitamins, Isopropyl stearate, Butyl stearate, Octyl palmitate, Cetyl palmitate, Tridecyl behenate, Diisopropyl adipate, Dioctyl sebacate, Menthyl anthranhilate, Cetyl octanoate, Octyl salicylate, Isopropyl myristate, neopentyl glycol dicarpate cetols, Ceraphyls®, Decyl oleate, diisopropyl adipate, C₁₂₋₁₅ alkyl lactates, Cetyl lactate, Lauryl lactate, Isostearyl neopentanoate, Myristyl lactate, Isocetyl stearoyl stearate, Octyldodecyl stearoyl stearate, Hydrocarbon oils, Isoparaffin, Fluid paraffins, Isododecane, Petrolatum, Argan oil, Canola oil, Chile oil, Coconut oil, corn oil, Cottonseed oil, Flaxseed oil, Grape seed oil, Mustard oil, Olive oil, Palm oil, Palm kernel oil, Peanut oil, Pine seed oil, Poppy seed oil, Pumpkin seed oil, Rice bran oil, Brand A Safflower oil, Tea oil, Truffle oil, Vegetable oil, Apricot (kernel) oil, Jojoba oil (simmondsia chinensis seed oil), Grapeseed oil, Macadamia oil, Wheat germ oil, Almond oil, Rapeseed oil, Gourd oil, Soybean oil, Sesame oil, Hazelnut oil, Maize oil, Sunflower oil, Hemp oil, Bois oil, Kuki nut oil, Avocado oil, Walnut oil, Fish oil, berry oil, allspice oil, juniper oil, seed oil, almond seed oil, anise seed oil, celery seed oil, cumin seed oil, nutmeg seed oil, leaf oil, basil leaf oil, bay leaf oil, cinnamon leaf oil, common sage leaf oil, eucalyptus leaf oil, lemon grass leaf oil, melaleuca leaf oil, oregano leaf oil, patchouli leaf oil, peppermint leaf oil, pine needle oil, rosemary leaf oil, spearmint leaf oil, tea tree leaf oil, thyme leaf oil, wintergreen leaf oil, flower oil, chamomile oil, clary sage oil, clove oil, geranium flower oil, hyssop flower oil, jasmine flower oil, lavender flower oil, manuka flower oil, Marhoram flower oil, orange flower oil, rose flower oil, ylang-ylang flower oil, Bark oil, cassia Bark oil, cinnamon bark oil, sassafras Bark oil, Wood oil, camphor wood oil, cedar wood oil, rosewood oil, sandalwood oil), rhizome (ginger) wood oil, resin oil, frankincense oil, myrrh oil, peel oil, bergamot peel oil, grapefruit peel oil, lemon peel oil, lime peel oil, orange peel oil, tangerine peel oil, root oil, valerian oil, Oleic acid, Linoleic acid, Oleyl alcohol, Isostearyl alcohol, semi-synthetic derivatives thereof, and any combinations thereof.

In some embodiments, the oil phase may comprise at least one medium chain triglyceride (MCT). MCTs are triglycerides whose fatty acids have an aliphatic tail of 6-12 carbon atoms. The fatty acids found in MCTs are called medium-chain fatty acids (MCFAs). Like all triglycerides, MCTs are composed of a glycerol backbone and three fatty acids. In the case of MCTs, 2 or 3 of the fatty acid chains attached to glycerol are of medium length. Common medium-chain fatty acids include caproic acid, caprlylic acid, capric acid, lauric acid, and sources commonly used for extraction of natural MCTs include palm kernal oil, and coconut oil.

In some embodiments, the oil phase may comprise a commercially available lipid or lipid mixture, such as various forms of BRAND A MEDIUM CHAIN TRIGLYCERIES, which can include but are not limited to an emollient ester, PPG-3 benzyl ether myristate, stearyl heptanoate, stearyl caprylate, propylene glycol dicaprylocaprate, and medium chain triglycerides (e.g., BRAND A MEDIUM CHAIN TRIGLYCERIES).

The oil may further comprise a silicone component, such as a volatile silicone component, which can be the sole oil in the silicone component or such silicone component can be combined with other silicone and non-silicone, volatile and non-volatile oils. Suitable silicone components include, but are not limited to, methylphenylpolysiloxane, simethicone, dimethicone, phenyltrimethicone (or an organomodified version thereof), alkylated derivatives of polymeric silicones, cetyl dimethicone, lauryl trimethicone, hydroxylated derivatives of polymeric silicones, such as dimethiconol, volatile silicone oils, cyclic and linear silicones, cyclomethicone, derivatives of cyclomethicone, hexamethylcyclotrisiloxane, octamethylcyclotetrasiloxane, decamethylcyclopentasiloxane, volatile linear dimethylpolysiloxanes, isohexadecane, isoeicosane, isotetracosane, polyisobutene, isooctane, isododecane, semi-synthetic derivatives thereof, and combinations thereof.

A volatile oil of the invention can be an organic solvent itself, or the volatile oil can be present in addition to an organic solvent. Suitable volatile oils include, but are not limited to, a terpene, monoterpene, sesquiterpene, carminative, azulene, menthol, camphor, thujone, thymol, nerol, linalool, limonene, geraniol, perillyl alcohol, nerolidol, farnesol, ylangene, bisabolol, farnesene, ascaridole, chenopodium oil, citronellal, citral, citronellol, chamazulene, yarrow, guaiazulene, chamomile, semi-synthetic derivatives, or combinations thereof.

The oil/lipid phase of the formulation may make up about 10% to about 70% (w/w) of the formulation, about 20% to about 50% (w/w) of the formulation, or about 30% to about 40% (w/w) of the formulation. For example, the oil phase may be about 10%, about 15%, about 20%, about 25%, about 30%, about 35%, about 40%, about 45%, about 50%, about 55%, or about 70% (w/w) of the formulation.

### Solvents

A nanoemulsion of the present disclosure is also not limited to a particular solvent, such as an organic solvent. A variety of solvents are contemplated including, but not limited to, an alcohol (e.g., including, but not limited to, methanol, ethanol, propanol, and octanol), glycerol, isopropyl myristate, triacetin, N-methyl pyrrolidinone, aliphatic or aromatic alcohols, and such solvents include polyethylene glycols, propylene glycol, and an organic phosphate based solvent.

Organic solvents that may be incorporated into the disclosed nanoemulsion formulations include, but are not limited to, C₁-C₁₂ alcohol, diol, triol, dialkyl phosphate, tri-alkyl phosphate, such as tri-n-butyl phosphate, semi-synthetic derivatives thereof, and combinations thereof. In some embodiments, the solvent may be a nonpolar solvent, a polar solvent, a protic solvent, or an aprotic solvent.

Suitable organic solvents for the disclosed nanoemulsion formulations may include, but are not limited to, ethanol, methanol, isopropyl alcohol, glycerol, medium chain triglycerides, diethyl ether, ethyl acetate, acetone, dimethyl sulfoxide (DMSO), acetic acid, n-butanol, butylene glycol, perfumers alcohols, isopropanol, n-propanol, formic acid, propylene glycols, glycerol, sorbitol, industrial methylated spirit, triacetin, hexane, benzene, toluene, diethyl ether, chloroform, 1,4-dixoane, tetrahydrofuran, dichloromethane, acetone, acetonitrile, dimethylformamide, dimethyl sulfoxide, formic acid, semi-synthetic derivatives thereof, and any combination thereof.

The solvent may make up about 0.05% to about 0.5% of the formulation, about 0.1% to about 0.4% of the formulation, or about 0.2% to about 0.3% of the formulation. For example, the solvent may be about 0.05%, about 0.1%, about 0.15%, about 0.2%, about 0.25%, about 0.3%, about 0.35%, about 0.4%, about 0.45%, or about 0.5% of the formulation.

### Surfactants

In some embodiments, the disclosed nanoemulsion formulations may further comprise a surfactant. The present disclosure is not limited to a particular surfactant. A variety of surfactants are contemplated including, but not limited to, nonionic and ionic surfactants (e.g., Brand A Span-80 polysorbate 20, polysorbate 80, and tyloxapol).

The surfactant in the disclosed nanoemulsion formulations can be a pharmaceutically acceptable ionic surfactant, a pharmaceutically acceptable nonionic surfactant, a pharmaceutically acceptable cationic surfactant, a pharmaceutically acceptable anionic surfactant, a pharmaceutically acceptable zwitterionic surfactant, or any combination thereof.

In some embodiments, the disclosed nanoemulsion formulations can comprise a cationic surfactant such as cetylpyridinium chloride (CPC).

In some embodiments, the disclosed nanoemulsion formulations comprises a nonionic surfactant, such as a polysorbate surfactant, which may be polysorbate 80 or polysorbate 20, and may have a concentration of about 1% to about 15%, (w/w) or about 5% to about 10% (w/w).

Exemplary useful surfactants are further described in Applied Surfactants: Principles and Application, Tharwat F. Tadros (Wiley-VCH Verlag Gmbh & Co., KGaA, Weinheim ISBN: 3-527-30629-3 (2005)). which is specifically incorporated by reference. Without being limited to type of surfactant, preferred surfactants are Poly-80, Poly-20 and Tween-80.

Useful surfactants can be a pharmaceutically acceptable ionic polymeric surfactant, a pharmaceutically acceptable nonionic polymeric surfactant, a pharmaceutically acceptable cationic polymeric surfactant, a pharmaceutically acceptable anionic polymeric surfactant, or a pharmaceutically acceptable zwitterionic polymeric surfactant. Examples of polymeric surfactants include, but are not limited to, a graft copolymer of a poly(methyl methacrylate) backbone with multiple (at least one) polyethylene oxide (PEO) side chain, polyhydroxystearic acid, an alkoxylated alkyl phenol formaldehyde condensate, a polyalkylene glycol modified polyester with hydrophobic fatty acids, a polyester, semi-synthetic derivatives thereof, or combinations thereof.

Surface active agents or surfactants are amphipathic molecules that consist of a non-polar hydrophobic portion, usually a straight or branched hydrocarbon or fluorocarbon chain containing 8-18 carbon atoms, attached to a polar or ionic hydrophilic portion. The hydrophilic portion can be nonionic, ionic or zwitterionic. The hydrocarbon chain interacts weakly with the water molecules in an aqueous environment, whereas the polar or ionic head group interacts strongly with water molecules via dipole or ion-dipole interactions. Based on the nature of the hydrophilic group, surfactants are classified into anionic, cationic, zwitterionic, nonionic and polymeric surfactants.

Suitable surfactants include, but are not limited to, ethoxylated nonylphenol comprising 9 to 10 units of ethyleneglycol, ethoxylated undecanol comprising 8 units of ethyleneglycol, polyoxyethylene (20) sorbitan monolaurate, polyoxyethylene (20) sorbitan monopalmitate, polyoxyethylene (20) sorbitan monostearate, polyoxyethylene (20) sorbitan monooleate, sorbitan monolaurate, sorbitan monopalmitate, sorbitan monostearate, sorbitan monooleate, ethoxylated hydrogenated ricin oils, sodium laurylsulfate, a diblock copolymer of ethyleneoxyde and propyleneoxyde, Ethylene Oxide-Propylene Oxide Block Copolymers, and tetra-functional block copolymers based on ethylene oxide and propylene oxide, Glyceryl monoesters, Glyceryl caprate, Glyceryl caprylate, Glyceryl cocate, Glyceryl erucate, Glyceryl hydroxysterate, Glyceryl isostearate, Glyceryl lanolate, Glyceryl laurate, Glyceryl linolate, Glyceryl myristate, Glyceryl oleate, Glyceryl PABA, Glyceryl palmitate, Glyceryl ricinoleate, Glyceryl stearate, Glyceryl thiglycolate, Glyceryl dilaurate, Glyceryl dioleate, Glyceryl dimyristate, Glyceryl disterate, Glyceryl sesuioleate, Glyceryl stearate lactate, Polyoxyethylene cetyl/stearyl ether, Polyoxyethylene cholesterol ether, Polyoxyethylene laurate or dilaurate, Polyoxyethylene stearate or distearate, polyoxyethylene fatty ethers, Polyoxyethylene lauryl ether, Polyoxyethylene stearyl ether, polyoxyethylene myristyl ether, a steroid, Cholesterol, Betasitosterol, Bisabolol, fatty acid esters of alcohols, isopropyl myristate, Aliphati-isopropyl n-butyrate, Isopropyl n-hexanoate, Isopropyl n-decanoate, Isoproppyl palmitate, Octyldodecyl myristate, alkoxylated alcohols, alkoxylated acids, alkoxylated amides, alkoxylated sugar derivatives, alkoxylated derivatives of natural oils and waxes, polyoxyethylene polyoxypropylene block copolymers, nonoxynol-14, PEG-8 laurate, PEG-6 Cocoamide, PEG-20 methylglucose sesquistearate, PEG40 lanolin, PEG-40 castor oil, PEG-40 hydrogenated castor oil, polyoxyethylene fatty ethers, glyceryl diesters, polyoxyethylene stearyl ether, polyoxyethylene myristyl ether, and polyoxyethylene lauryl ether, glyceryl dilaurate, glyceryl dimystate, glyceryl distearate, semi-synthetic derivatives thereof, or mixtures thereof.

Additional suitable surfactants include, but are not limited to, non-ionic lipids, such as glyceryl laurate, glyceryl myristate, glyceryl dilaurate, glyceryl dimyristate, semi-synthetic derivatives thereof, and mixtures thereof.

In additional embodiments, the surfactant is a polyoxyethylene fatty ether having a polyoxyethylene head group ranging from about 2 to about 100 groups, or an alkoxylated alcohol having the structure R₅—(OCH₂CH₂)y—OH, wherein R₅ is a branched or unbranched alkyl group having from about 6 to about 22 carbon atoms and y is between about 4 and about 100, and preferably, between about 10 and about 100. Preferably, the alkoxylated alcohol is the species wherein R₅ is a lauryl group and y has an average value of 23.

In a different embodiment, the surfactant is an alkoxylated alcohol which is an ethoxylated derivative of lanolin alcohol. Preferably, the ethoxylated derivative of lanolin alcohol is laneth-10, which is the polyethylene glycol ether of lanolin alcohol with an average ethoxylation value of 10.

Nonionic surfactants include, but are not limited to, an ethoxylated surfactant, an alcohol ethoxylated, an alkyl phenol ethoxylated, a fatty acid ethoxylated, a monoalkaolamide ethoxylated, a sorbitan ester ethoxylated, a fatty amino ethoxylated, an ethylene oxide-propylene oxide copolymer, Bis(polyethylene glycol bis[imidazoyl carbonyl]), nonoxynol-9, Bis(polyethylene glycol bis[imidazoyl carbonyl]), Brij® 35, Brij® 56, Brij® 72, Brij® 76, Brij® 92V, Brij® 97, Brij® 58P, Cremophor® EL, Decaethylene glycol monododecyl ether, N-Decanoyl-N-methylglucamine, n-Decyl alpha-D-glucopyranoside, Decyl beta-D-maltopyranoside, n-Dodecanoyl-N-methylglucamide, n-Dodecyl alpha-D-maltoside, n-Dodecyl beta-D-maltoside, n-Dodecyl beta-D-maltoside, Heptaethylene glycol monodecyl ether, Heptaethylene glycol monododecyl ether, Heptaethylene glycol monotetradecyl ether, n-Hexadecyl beta-D-maltoside, Hexaethylene glycol monododecyl ether, Hexaethylene glycol monohexadecyl ether, Hexaethylene glycol monooctadecyl ether, Hexaethylene glycol monotetradecyl ether, Igepal CA-630, Igepal CA-630, Methyl-6-O-(N-heptylcarbamoyl)-alpha-D-glucopyranoside, Nonaethylene glycol monododecyl ether, N-Nonanoyl-N-methylglucamine, N-Nonanoyl-N-methylglucamine, Octaethylene glycol monodecyl ether, Octaethylene glycol monododecyl ether, Octaethylene glycol monohexadecyl ether, Octaethylene glycol monooctadecyl ether, Octaethylene glycol monotetradecyl ether, Octyl-beta-D-glucopyranoside, Pentaethylene glycol monodecyl ether, Pentaethylene glycol monododecyl ether, Pentaethylene glycol monohexadecyl ether, Pentaethylene glycol monohexyl ether, Pentaethylene glycol monooctadecyl ether, Pentaethylene glycol monooctyl ether, Polyethylene glycol diglycidyl ether, Polyethylene glycol ether W-1, Polyoxyethylene 10 tridecyl ether, Polyoxyethylene 100 stearate, Polyoxyethylene 20 isohexadecyl ether, Polyoxyethylene 20 oleyl ether, Polyoxyethylene 40 stearate, Polyoxyethylene 50 stearate, Polyoxyethylene 8 stearate, Polyoxyethylene bis(imidazolyl carbonyl), Polyoxyethylene 25 propylene glycol stearate, Saponin from Quillaja bark, Span® 20, Span® 40, Span® 60, Span® 65, Span® 80, Span® 85, Tergitol, Type 15-S-12, Tergitol, Type 15-S-30, Tergitol, Type 15-S-5, Tergitol, Type 15-S-7, Tergitol, Type 15-S-9, Tergitol, Type NP-10, Tergitol, Type NP-4, Tergitol, Type NP-40, Tergitol, Type NP-7, Tergitol, Type NP-9, Tergitol, Tergitol, Type TMN-10, Tergitol, Type TMN-6, Tetradecyl-beta-D-maltoside, Tetraethylene glycol monodecyl ether, Tetraethylene glycol monododecyl ether, Tetraethylene glycol monotetradecyl ether, Triethylene glycol monodecyl ether, Triethylene glycol monododecyl ether, Triethylene glycol monohexadecyl ether, Triethylene glycol monooctyl ether, Triethylene glycol monotetradecyl ether, Triton® CF-21, Triton® CF-32, Triton® DF-12, Triton® DF-16, Triton® GR-5M, Triton® QS-15, Triton® QS-44, Triton® X-100, Triton® X-102, Triton® X-15, Triton® X-151, Triton® X-200, Triton® X-207, Triton® X-100, Triton® X-114, Triton® X-165, Triton® X-305, Triton® X-405, Triton® X-45, Triton® X-705-70, Tween® 20, Tween® 21, Tween® 40, Tween® 60, Tween® 61, Tween® 65, Tween® 80, Tween® 81, Tween® 85, Tyloxapol, n-Undecyl beta-D-glucopyranoside, semi-synthetic derivatives thereof, or combinations thereof.

In addition, the nonionic surfactant can be a poloxamer. Poloxamers are polymers made of a block of polyoxyethylene, followed by a block of polyoxypropylene, followed by a block of polyoxyethylene. The average number of units of polyoxyethylene and polyoxypropylene varies based on the number associated with the polymer. For example, the smallest polymer, Poloxamer 101, consists of a block with an average of 2 units of polyoxyethylene, a block with an average of 16 units of polyoxypropylene, followed by a block with an average of 2 units of polyoxyethylene. Poloxamers range from colorless liquids and pastes to white solids. In cosmetics and personal care products, poloxamers are used in the formulation of skin cleansers, bath products, shampoos, hair conditioners, mouthwashes, eye makeup remover and other skin and hair products. Examples of Poloxamers include, but are not limited to, Poloxamer 101, Poloxamer 105, Poloxamer 108, Poloxamer 122, Poloxamer 123, Poloxamer 124, Poloxamer 181, Poloxamer 182, Poloxamer 183, Poloxamer 184, Poloxamer 185, Poloxamer 188, Poloxamer 212, Poloxamer 215, Poloxamer 217, Poloxamer 231, Poloxamer 234, Poloxamer 235, Poloxamer 237, Poloxamer 238, Poloxamer 282, Poloxamer 284, Poloxamer 288, Poloxamer 331, Poloxamer 333, Poloxamer 334, Poloxamer 335, Poloxamer 338, Poloxamer 401, Poloxamer 402, Poloxamer 403, Poloxamer 407, Poloxamer 105 Benzoate, and Poloxamer 182 Dibenzoate.

Suitable cationic surfactants include, but are not limited to, a quarternary ammonium compound, an alkyl trimethyl ammonium chloride compound, a dialkyl dimethyl ammonium chloride compound, a cationic halogen-containing compound, such as cetylpyridinium chloride, Benzalkonium chloride, Benzalkonium chloride, Benzyldimethylhexadecylammonium chloride, Benzyldimethyltetradecylammonium chloride, Benzyldodecyldimethylammonium bromide, Benzyltrimethylammonium tetrachloroiodate, Dimethyldioctadecylammonium bromide, Dodecylethyldimethylammonium bromide, Dodecyltrimethylammonium bromide, Dodecyltrimethylammonium bromide, Ethylhexadecyldimethylammonium bromide, Girard's reagent T, Hexadecyltrimethylammonium bromide, Hexadecyltrimethylammonium bromide, N,N′,N′-Polyoxyethylene(10)-N-tallow-1,3-diaminopropane, Thonzonium bromide, Trimethyl(tetradecyl)ammonium bromide, 1,3,5-Triazine-1,3,5(2H,4H,6H)-triethanol, 1-Decanaminium, N-decyl-N, N-dimethyl-, chloride, Didecyl dimethyl ammonium chloride, 2-(2-(p-(Diisobutyl)cresosxy)ethoxy)ethyl dimethyl benzyl ammonium chloride, 2-(2-(p-(Diisobutyl)phenoxy)ethoxy)ethyl dimethyl benzyl ammonium chloride, Alkyl 1 or 3 benzyl-1-(2-hydroxethyl)-2-imidazolinium chloride, Alkyl bis(2-hydroxyethyl) benzyl ammonium chloride, Alkyl demethyl benzyl ammonium chloride, Alkyl dimethyl 3,4-dichlorobenzyl ammonium chloride (100% C₁₂), Alkyl dimethyl 3,4-dichlorobenzyl ammonium chloride (50% C₁₄, 40% C₁₂, 10% C₁₆), Alkyl dimethyl 3,4-dichlorobenzyl ammonium chloride (55% C₁₄, 23% C₁₂, 20% C₁₆), Alkyl dimethyl benzyl ammonium chloride, Alkyl dimethyl benzyl ammonium chloride (100% C₁₄), Alkyl dimethyl benzyl ammonium chloride (100% C₁₆), Alkyl dimethyl benzyl ammonium chloride (41% C₁₄, 28% C₁₂), Alkyl dimethyl benzyl ammonium chloride (47% C₁₂, 18% C₁₄), Alkyl dimethyl benzyl ammonium chloride (55% C₁₆, 20% C₁₄), Alkyl dimethyl benzyl ammonium chloride (58% C₁₄, 28% C₁₆), Alkyl dimethyl benzyl ammonium chloride (60% C₁₄, 25% C₁₂), Alkyl dimethyl benzyl ammonium chloride (61% C₁₁, 23% C₁₄), Alkyl dimethyl benzyl ammonium chloride (61% C₁₂, 23% C₁₄), Alkyl dimethyl benzyl ammonium chloride (65% C₁₂, 25% C₁₄), Alkyl dimethyl benzyl ammonium chloride (67% C₁₂, 24% C₁₄), Alkyl dimethyl benzyl ammonium chloride (67% C₁₂, 25% C₁₄), Alkyl dimethyl benzyl ammonium chloride (90% C₁₄, 5% C₁₂), Alkyl dimethyl benzyl ammonium chloride (93% C₁₄, 4% C₁₂), Alkyl dimethyl benzyl ammonium chloride (95% C₁₆, 5% C₁₈), Alkyl dimethyl benzyl ammonium chloride, Alkyl didecyl dimethyl ammonium chloride, Alkyl dimethyl benzyl ammonium chloride, Alkyl dimethyl benzyl ammonium chloride (C₁₂₋₁₆), Alkyl dimethyl benzyl ammonium chloride (C₁₂₋₁₈), Alkyl dimethyl benzyl ammonium chloride, dialkyl dimethyl benzyl ammonium chloride, Alkyl dimethyl dimethybenzyl ammonium chloride, Alkyl dimethyl ethyl ammonium bromide (90% C₁₄, 5% C₁₆, 5% C₁₂), Alkyl dimethyl ethyl ammonium bromide (mixed alkyl and alkenyl groups as in the fatty acids of soybean oil), Alkyl dimethyl ethylbenzyl ammonium chloride, Alkyl dimethyl ethylbenzyl ammonium chloride (60% C₁₄), Alkyl dimethyl isopropylbenzyl ammonium chloride (50% C₁₂, 30% C₁₄, 17% C₁₆, 3% C₁₈), Alkyl trimethyl ammonium chloride (58% C₁₈, 40% C₁₆, 1% C₁₄, 1% C₁₂), Alkyl trimethyl ammonium chloride (90% C₁₈, 10% C₁₆), Alkyldimethyl(ethylbenzyl) ammonium chloride (C₁₂₋₁₈), Di-(C₈₋₁₀)-alkyl dimethyl ammonium chlorides, Dialkyl dimethyl ammonium chloride, Dialkyl methyl benzyl ammonium chloride, Didecyl dimethyl ammonium chloride, Diisodecyl dimethyl ammonium chloride, Dioctyl dimethyl ammonium chloride, Dodecyl bis (2-hydroxyethyl) octyl hydrogen ammonium chloride, Dodecyl dimethyl benzyl ammonium chloride, Dodecylcarbamoyl methyl dinethyl benzyl ammonium chloride, Heptadecyl hydroxyethylimidazolinium chloride, Hexahydro-1,3,5-tris(2-hydroxyethyl)-s-triazine, Hexahydro-1,3,5-tris(2-hydroxyethyl)-s-triazine, Myristalkonium chloride (and) Quat RNIUM 14, N,N-Dimethyl-2-hydroxypropylammonium chloride polymer, n-Tetradecyl dimethyl benzyl ammonium chloride monohydrate, Octyl decyl dimethyl ammonium chloride, Octyl dodecyl dimethyl ammonium chloride, Octyphenoxyethoxyethyl dimethyl benzyl ammonium chloride, Oxydiethylenebis(alkyl dimethyl ammonium chloride), Quaternary ammonium compounds, dicoco alkyldimethyl, chloride, Trimethoxysily propyl dimethyl octadecyl ammonium chloride, Trimethoxysilyl quats, Trimethyl dodecylbenzyl ammonium chloride, semi-synthetic derivatives thereof, and combinations thereof.

Exemplary cationic halogen-containing compounds include, but are not limited to, cetylpyridinium halides, cetyltrimethylammonium halides, cetyldimethylethylammonium halides, cetyldimethylbenzylammonium halides, cetyltributylphosphonium halides, dodecyltrimethylammonium halides, or tetradecyltrimethylammonium halides. In some particular embodiments, suitable cationic halogen containing compounds comprise, but are not limited to, cetylpyridinium chloride (CPC), cetyltrimethylammonium chloride, cetylbenzyldimethylammonium chloride, cetylpyridinium bromide (CPB), cetyltrimethylammonium bromide (CTAB), cetyidimethylethylammonium bromide, cetyltributylphosphonium bromide, dodecyltrimethylammonium bromide, and tetrad ecyltrimethylammonium bromide. In particularly preferred embodiments, the cationic halogen containing compound is CPC, although the compositions of the present invention are not limited to formulation with a particular cationic containing compound.

Suitable anionic surfactants include, but are not limited to, a carboxylate, a sulphate, a sulphonate, a phosphate, chenodeoxycholic acid, chenodeoxycholic acid sodium salt, cholic acid, ox or sheep bile, Dehydrocholic acid, Deoxycholic acid, Deoxycholic acid, Deoxycholic acid methyl ester, Digitonin, Digitoxigenin, N,N-Dimethyldodecylamine N-oxide, Docusate sodium salt, Glycochenodeoxycholic acid sodium salt, Glycocholic acid hydrate, synthetic, Glycocholic acid sodium salt hydrate, synthetic, Glycodeoxycholic acid monohydrate, Glycodeoxycholic acid sodium salt, Glycodeoxycholic acid sodium salt, Glycolithocholic acid 3-sulfate disodium salt, Glycolithocholic acid ethyl ester, N-Lauroylsarcosine sodium salt, N-Lauroylsarcosine solution, N-Lauroylsarcosine solution, Lithium dodecyl sulfate, Lithium dodecyl sulfate, Lithium dodecyl sulfate, Lugol solution, Niaproof 4, Type 4, 1-Octanesulfonic acid sodium salt, Sodium 1-butanesulfonate, Sodium 1-decanesulfonate, Sodium 1-decanesulfonate, Sodium 1-dodecanesulfonate, Sodium 1-heptanesulfonate anhydrous, Sodium 1-heptanesulfonate anhydrous, Sodium 1-nonanesulfonate, Sodium 1-propanesulfonate monohydrate, Sodium 2-bromoethanesulfonate, Sodium cholate hydrate, Sodium choleate, Sodium deoxycholate, Sodium deoxycholate monohydrate, Sodium dodecyl sulfate, Sodium hexanesulfonate anhydrous, Sodium octyl sulfate, Sodium pentanesulfonate anhydrous, Sodium taurocholate, Taurochenodeoxycholic acid sodium salt, Taurodeoxycholic acid sodium salt monohydrate, Taurohyodeoxycholic acid sodium salt hydrate, Taurolithocholic acid 3-sulfate disodium salt, Tauroursodeoxycholic acid sodium salt, Trizma® dodecyl sulfate, Tween® 80, Ursodeoxycholic acid, semi-synthetic derivatives thereof, and combinations thereof.

Suitable zwitterionic surfactants include, but are not limited to, an N-alkyl betaine, lauryl amindo propyl dimethyl betaine, an alkyl dimethyl glycinate, an N-alkyl amino propionate, CHAPS, minimum 98% (TLC), CHAPS, SigmaUltra, minimum 98% (TLC), CHAPS, for electrophoresis, minimum 98% (TLC), CHAPSO, minimum 98%, CHAPSO, SigmaUltra, CHAPSO, for electrophoresis, 3-(Decyldimethylammonio)propanesulfonate inner salt, 3-Dodecyldimethylammonio)propanesulfonate inner salt, SigmaUltra, 3-(Dodecyldimethylammonio)propanesulfonate inner salt, 3-(N,N-Dimethylmyristylammonio)propanesulfonate, 3-(N,N-Dimethyloctadecylammonio)propanesulfonate, 3-(N,N-Dimethyloctylammonio)propanesulfonate inner salt, 3-(N,N-Dimethylpalmitylammonio)propanesulfonate, semi-synthetic derivatives thereof, and combinations thereof.

In some embodiments, the disclosed nanoemulsion formulations comprises a surfactant in a concentration of about 1% to about 15%, about 3% to about 12%, or about 5% to about 10% (w/w). For example, disclosed nanoemulsion formulations may comprise about 1, about 2, about 3, about 4, about 5, about 6, about 7, about 8, about 9, about 10, about 11, about 12, about 13, about 14, or about 15% (w/w) of a suitable surfactant (e.g., a polysorbate or a polyethylene glycol).

### Additional Components

Additional compounds suitable for use in the disclosed nanoemulsion formulations may include but are not limited to one or more solvents, such as an organic phosphate-based solvent, bulking agents, coloring agents, pharmaceutically acceptable excipients, a preservative, pH adjuster, buffer, chelating agent, etc. The additional compounds can be admixed into a previously emulsified nanoemulsion, or the additional compounds can be added to the original mixture to be emulsified. In certain of these embodiments, one or more additional compounds are admixed into an existing nanoemulsion composition immediately prior to its use.

Suitable preservatives in the disclosed nanoemulsion formulations include, but are not limited to, benzalkonium chloride (BKC), methyl paraben, benzyl alcohol, cetylpyridinium chloride, chlorhexidine, imidazolidinyl urea, phenol, potassium sorbate, benzoic acid, bronopol, chlorocresol, paraben esters, phenoxyethanol, sorbic acid, alpha-tocophernol, ascorbic acid, ascorbyl palmitate, butylated hydroxyanisole, butylated hydroxytoluene, sodium ascorbate, sodium metabisulphite, citric acid, edetic acid, semi-synthetic derivatives thereof, and combinations thereof. Other suitable preservatives include, but are not limited to, benzyl alcohol, chlorhexidine (bis (p-chlorophenyldiguanido) hexane), chlorphenesin (3-(-4-chloropheoxy)-propane-1,2-diol), Kathon CG (methyl and methylchloroisothiazolinone), parabens (methyl, ethyl, propyl, butyl hydrobenzoates), phenoxyethanol (2-phenoxyethanol), sorbic acid (potassium sorbate, sorbic acid), Phenonip (phenoxyethanol, methyl, ethyl, butyl, propyl parabens), Phenoroc (phenoxyethanol 0.73%, methyl paraben 0.2%, propyl paraben 0.07%), Liquipar Oil (isopropyl, isobutyl, butylparabens), Liquipar PE (70% phenoxyethanol, 30% liquipar oil), Nipaguard MPA (benzyl alcohol (70%), methyl & propyl parabens), Nipaguard MPS (propylene glycol, methyl & propyl parabens), Nipasept (methyl, ethyl and propyl parabens), Nipastat (methyl, butyl, ethyl and propyel parabens), Elestab 388 (phenoxyethanol in propylene glycol plus chlorphenesin and methylparaben), and Killitol (7.5% chlorphenesin and 7.5% methyl parabens). In some embodiments, the nanoemulsion formulations may include more than one of the disclosed preservatives.

The disclosed nanoemulsion formulations may further comprise at least one pH adjuster. Suitable pH adjusters in the nanoemulsion vaccine of the invention include, but are not limited to, diethyanolamine, lactic acid, monoethanolamine, triethylanolamine, sodium hydroxide, sodium phosphate, semi-synthetic derivatives thereof, and combinations thereof.

In addition, the disclosed nanoemulsion formulations can comprise a chelating agent. In one embodiment of the invention, the chelating agent is present in an amount of about 0.0005% to about 1%. Examples of chelating agents include, but are not limited to, ethylenediamine, ethylenediaminetetraacetic acid (EDTA), phytic acid, polyphosphoric acid, citric acid, gluconic acid, acetic acid, lactic acid, and dimercaprol, and a preferred chelating agent is ethylenediaminetetraacetic acid.

The disclosed nanoemulsion formulations can comprise a buffering agent, such as a pharmaceutically acceptable buffering agent. Examples of buffering agents include, but are not limited to, 2-Amino-2-methyl-1,3-propanediol, ≥99.5% (NT), 2-Amino-2-methyl-1-propanol, ≥99.0% (GC), L-(+)-Tartaric acid, ≥99.5% (T), ACES, ≥99.5% (T), ADA, ≥99.0% (T), Acetic acid, ≥99.5% (GC/T), Acetic acid, for luminescence, ≥99.5% (GC/T), Ammonium acetate solution, for molecular biology, ˜5 M in H₂O, Ammonium acetate, for luminescence, ≥99.0% (calc. on dry substance, T), Ammonium bicarbonate, ≥99.5% (T), Ammonium citrate dibasic, ≥99.0% (T), Ammonium formate solution, 10 M in H2O, Ammonium formate, ≥99.0% (calc. based on dry substance, NT), Ammonium oxalate monohydrate, ≥99.5% (RT), Ammonium phosphate dibasic solution, 2.5 M in H₂O, Ammonium phosphate dibasic, ≥99.0% (T), Ammonium phosphate monobasic solution, 2.5 M in H₂O, Ammonium phosphate monobasic, ≥99.5% (T), Ammonium sodium phosphate dibasic tetrahydrate, ≥99.5% (NT), Ammonium sulfate solution, for molecular biology, 3.2 M in H₂O, Ammonium tartrate dibasic solution, 2 M in H2O (colorless solution at 20° C.), Ammonium tartrate dibasic, ≥99.5% (T), BES buffered saline, for molecular biology, 2× concentrate, BES, ≥99.5% (T), BES, for molecular biology, ≥99.5% (T), BICINE buffer Solution, for molecular biology, 1 M in H₂O, BICINE, ≥99.5% (T), BIS-TRIS, ≥99.0% (NT), Bicarbonate buffer solution, >0.1 M Na2CO₃, >0.2 M NaHCO₃, Boric acid, ≥99.5% (T), Boric acid, for molecular biology, ≥99.5% (T), CAPS, ≥99.0% (TLC), CHES, ≥99.5% (T), Calcium acetate hydrate, ≥99.0% (calc. on dried material, KT), Calcium carbonate, precipitated, ≥99.0% (KT), Calcium citrate tribasic tetrahydrate, ≥98.0% (calc. on dry substance, KT), Citrate Concentrated Solution, for molecular biology, 1 M in H₂O, Citric acid, anhydrous, ≥99.5% (T), Citric acid, for luminescence, anhydrous, ≥99.5% (T), Diethanolamine, ≥99.5% (GC), EPPS, ≥99.0% (T), Ethylenediaminetetraacetic acid disodium salt dihydrate, for molecular biology, ≥99.0% (T), Formic acid solution, 1.0 M in H₂O, Gly-Gly-Gly, ≥99.0% (NT), Gly-Gly, ≥99.5% (NT), Glycine, ≥99.0% (NT), Glycine, for luminescence, ≥99.0% (NT), Glycine, for molecular biology, ≥99.0% (NT), HEPES buffered saline, for molecular biology, 2× concentrate, HEPES, ≥99.5% (T), HEPES, for molecular biology, ≥99.5% (T), Imidazole buffer Solution, 1 M in H₂O, Imidazole, ≥99.5% (GC), Imidazole, for luminescence, ≥99.5% (GC), Imidazole, for molecular biology, ≥99.5% (GC), Lipoprotein Refolding Buffer, Lithium acetate dihydrate, ≥99.0% (NT), Lithium citrate tribasic tetrahydrate, ≥99.5% (NT), MES hydrate, ≥99.5% (T), MES monohydrate, for luminescence, ≥99.5% (T), MES solution, for molecular biology, 0.5 M in H₂O, MOPS, ≥99.5% (T), MOPS, for luminescence, ≥99.5% (T), MOPS, for molecular biology, ≥99.5% (T), Magnesium acetate solution, for molecular biology, ˜1 M in H₂O, Magnesium acetate tetrahydrate, ≥99.0% (KT), Magnesium citrate tribasic nonahydrate, ≥98.0% (calc. based on dry substance, KT), Magnesium formate solution, 0.5 M in H₂O, Magnesium phosphate dibasic trihydrate, ≥98.0% (KT), Neutralization solution for the in-situ hybridization for in-situ hybridization, for molecular biology, Oxalic acid dihydrate, ≥99.5% (RT), PIPES, ≥99.5% (T), PIPES, for molecular biology, ≥99.5% (T), Phosphate buffered saline, solution (autoclaved), Phosphate buffered saline, washing buffer for peroxidase conjugates in Western Blotting, 10× concentrate, Piperazine, anhydrous, ≥99.0% (T), Potassium D-tartrate monobasic, ≥99.0% (T), Potassium acetate solution, for molecular biology, Potassium acetate solution, for molecular biology, 5 M in H₂O, Potassium acetate solution, for molecular biology, ˜1 M in H₂O, Potassium acetate, ≥99.0% (NT), Potassium acetate, for luminescence, ≥99.0% (NT), Potassium acetate, for molecular biology, ≥99.0% (NT), Potassium bicarbonate, ≥99.5% (T), Potassium carbonate, anhydrous, ≥99.0% (T), Potassium chloride, ≥99.5% (AT), Potassium citrate monobasic, ≥99.0% (dried material, NT), Potassium citrate tribasic solution, 1 M in H₂O, Potassium formate solution, 14 M in H₂O, Potassium formate, ≥99.5% (NT), Potassium oxalate monohydrate, ≥99.0% (RT), Potassium phosphate dibasic, anhydrous, ≥99.0% (T), Potassium phosphate dibasic, for luminescence, anhydrous, ≥99.0% (T), Potassium phosphate dibasic, for molecular biology, anhydrous, ≥99.0% (T), Potassium phosphate monobasic, anhydrous, ≥99.5% (T), Potassium phosphate monobasic, for molecular biology, anhydrous, ≥99.5% (T), Potassium phosphate tribasic monohydrate, ≥95% (T), Potassium phthalate monobasic, ≥99.5% (T), Potassium sodium tartrate solution, 1.5 M in H₂O, Potassium sodium tartrate tetrahydrate, ≥99.5% (NT), Potassium tetraborate tetrahydrate, ≥99.0% (T), Potassium tetraoxalate dihydrate, ≥99.5% (RT), Propionic acid solution, 1.0 M in H₂O, STE buffer solution, for molecular biology, pH 7.8, STET buffer solution, for molecular biology, pH 8.0, Sodium 5,5-diethylbarbiturate, ≥99.5% (NT), Sodium acetate solution, for molecular biology, ˜3 M in H₂O, Sodium acetate trihydrate, ≥99.5% (NT), Sodium acetate, anhydrous, ≥99.0% (NT), Sodium acetate, for luminescence, anhydrous, ≥99.0% (NT), Sodium acetate, for molecular biology, anhydrous, ≥99.0% (NT), Sodium bicarbonate, ≥99.5% (T), Sodium bitartrate monohydrate, ≥99.0% (T), Sodium carbonate decahydrate, ≥99.5% (T), Sodium carbonate, anhydrous, ≥99.5% (calc. on dry substance, T), Sodium citrate monobasic, anhydrous, ≥99.5% (T), Sodium citrate tribasic dihydrate, ≥99.0% (NT), Sodium citrate tribasic dihydrate, for luminescence, ≥99.0% (NT), Sodium citrate tribasic dihydrate, for molecular biology, ≥99.5% (NT), Sodium formate solution, 8 M in H₂O, Sodium oxalate, ≥99.5% (RT), Sodium phosphate dibasic dihydrate, ≥99.0% (T), Sodium phosphate dibasic dihydrate, for luminescence, ≥99.0% (T), Sodium phosphate dibasic dihydrate, for molecular biology, ≥99.0% (T), Sodium phosphate dibasic dodecahydrate, ≥99.0% (T), Sodium phosphate dibasic solution, 0.5 M in H₂O, Sodium phosphate dibasic, anhydrous, ≥99.5% (T), Sodium phosphate dibasic, for molecular biology, ≥99.5% (T), Sodium phosphate monobasic dihydrate, ≥99.0% (T), Sodium phosphate monobasic dihydrate, for molecular biology, ≥99.0% (T), Sodium phosphate monobasic monohydrate, for molecular biology, ≥99.5% (T), Sodium phosphate monobasic solution, 5 M in H₂O, Sodium pyrophosphate dibasic, ≥99.0% (T), Sodium pyrophosphate tetrabasic decahydrate, ≥99.5% (T), Sodium tartrate dibasic dihydrate, ≥99.0% (NT), Sodium tartrate dibasic solution, 1.5 M in H₂O (colorless solution at 20° C.), Sodium tetraborate decahydrate, ≥99.5% (T), TAPS, ≥99.5% (T), TES, ≥99.5% (calc. based on dry substance, T), TM buffer solution, for molecular biology, pH 7.4, TNT buffer solution, for molecular biology, pH 8.0, TRIS Glycine buffer solution, 10× concentrate, TRIS acetate-EDTA buffer solution, for molecular biology, TRIS buffered saline, 10× concentrate, TRIS glycine SDS buffer solution, for electrophoresis, 10× concentrate, TRIS phosphate-EDTA buffer solution, for molecular biology, concentrate, 10× concentrate, Tricine, ≥99.5% (NT), Triethanolamine, ≥99.5% (GC), Triethylamine, ≥99.5% (GC), Triethylammonium acetate buffer, volatile buffer, ˜1.0 M in H₂O, Triethylammonium phosphate solution, volatile buffer, ˜1.0 M in H₂O, Trimethylammonium acetate solution, volatile buffer, ˜1.0 M in H₂O, Trimethylammonium phosphate solution, volatile buffer, ˜1 M in H₂O, Tris-EDTA buffer solution, for molecular biology, concentrate, 100× concentrate, Tris-EDTA buffer solution, for molecular biology, pH 7.4, Tris-EDTA buffer solution, for molecular biology, pH 8.0, Trizma® acetate, ≥99.0% (NT), Trizma® base, ≥99.8% (T), Trizma® base, ≥99.8% (T), Trizma® base, for luminescence, ≥99.8% (T), Trizma® base, for molecular biology, ≥99.8% (T), Trizma® carbonate, ≥98.5% (T), Trizma® hydrochloride buffer solution, for molecular biology, pH 7.2, Trizma® hydrochloride buffer solution, for molecular biology, pH 7.4, Trizma® hydrochloride buffer solution, for molecular biology, pH 7.6, Trizma® hydrochloride buffer solution, for molecular biology, pH 8.0, Trizma® hydrochloride, ≥99.0% (AT), Trizma® hydrochloride, for luminescence, ≥99.0% (AT), Trizma® hydrochloride, for molecular biology, ≥99.0% (AT), and Trizma® maleate, ≥99.5% (NT).

The disclosed nanoemulsion formulations can comprise one or more emulsifying agents to aid in the formation of emulsions. Emulsifying agents include compounds that aggregate at the oil/water interface to form a kind of continuous membrane that prevents direct contact between two adjacent droplets.

### Globule and Particle Size

The disclosed nanoemulsion formulations may comprise droplets (i.e., globule of the oil phase) having an average diameter size of less than about 1,000 nm (i.e., 1 micron). In other embodiments of the invention, the droplet size has an average diameter of less than about 950 nm, less than about 900 nm, less than about 850 nm, less than about 800 nm, less than about 750 nm, less than about 700 nm, less than about 650 nm, less than about 600 nm, less than about 550 nm, less than about 500 nm, less than about 450 nm, less than about 400 nm, less than about 350 nm, less than about 300 nm, less than about 250 nm, less than about 200 nm, less than about 150 nm, or any combination thereof. In one embodiment, the droplets have an average diameter size greater than about 125 nm and less than or equal to about 600 nm. In a different embodiment, the droplets have an average diameter size greater than about 50 nm or greater than about 70 nm, and less than or equal to about 125 nm, less than or equal to about 130 nm, less than or equal to about 135 nm, less than or equal to about 140 nm, or less than or equal to about 145.

The disclosed nanoemulsion formulations may comprise particle of an active compound (e.g., THC, CBD, or CBDi, Diclofenac sodium, etc.) having an average diameter size of less than about 1,000 nm (i.e., 1 micron). In other embodiments of the invention, the active agent particle size has an average diameter of less than about 950 nm, less than about 900 nm, less than about 850 nm, less than about 800 nm, less than about 750 nm, less than about 700 nm, less than about 650 nm, less than about 600 nm, less than about 550 nm, less than about 500 nm, less than about 450 nm, less than about 400 nm, less than about 350 nm, less than about 300 nm, less than about 250 nm, less than about 200 nm, less than about 150 nm, or any combination thereof. In one embodiment, the droplets have an average diameter size greater than about 125 nm and less than or equal to about 600 nm. In a different embodiment, the active agent particles have an average diameter size greater than about 50 nm or greater than about 70 nm, and less than or equal to about 125 nm, less than or equal to about 130 nm, less than or equal to about 135 nm, less than or equal to about 140 nm, or less than or equal to about 145.

### Pharmaceutical Compositions

The disclosed nanoemulsion formulations may be formulated into pharmaceutical compositions that comprise the nanoemulsion in a therapeutically effective amount and suitable, pharmaceutically acceptable excipients for pharmaceutically acceptable delivery. Such excipients are well known in the art.

Exemplary dosage forms for pharmaceutical administration include, but are not limited to, drops, liquids, ointments, creams, emulsions, lotions, gels, bioadhesive gels, sprays, aerosols, pastes, foams, and suspensions. The disclosed formulations are generally intended for topical applications, which includes arthritis administration directly into or onto the eye.

The disclosed nanoemulsion formulations may be formulated for immediate release, sustained release, controlled release, delayed release, or any combinations thereof, into the epidermis or dermis. In some embodiments, the formulations may comprise a penetration- enhancing agent. Suitable penetration-enhancing agents include, but are not limited to, alcohols such as ethanol, triglycerides and aloe compositions.

The disclosed nanoemulsion formulations and pharmaceutical dosage forms thereof may be applied in a single administration or in multiple administrations. For instance, 1, 2, 3, 4, 5, 6, 7, 8, 9, or 10 or more times per day, every other day, every three days, every four days, every five days, every six days, or every week.

In some embodiments, the disclosed nanoemulsion formulations may be administered at total dosage amounts of about 10 mg to about 50,000 mg of the at least one cannabinoid or a salt or derivative thereof. In some embodiments, the disclosed nanoemulsion formulations may be administered at total dosage amounts of about 50 mg to about 5,000 mg of Diclofenac sodium or a salt or derivative thereof.

The dosage of the pharmaceutical composition can vary depending on a given patient's weight, age, gender, administration time and mode, and the severity of disease.

### Methods of Treating Arthritis Conditions

The present disclosure provides uses and methods for treating arthritis conditions, such as but not limited to joint pain, with nanoemulsions comprising Diclofenac sodium or a salt or derivative thereof optionally, at least one cannabinoid or a salt or derivative thereof (e.g., CBD or CBDi and/or THC).

In general, treatment of arthritis conditions such as joint pain will comprise topically administering one of the disclosed nanoemulsion formulations directly on the skin of a subject suffering from joint pain. Thus, the nanoemulsion formulations may commonly be prepared as a topical gel, hydrogel, cream, or other liquid formulation. When the nanoemulsion formulations are prepared in such a way, an exemplary dose may include about 1, about 2, about 3, about 4, about 5 or more drops of the gel, hydrogel, cream, suspension, or other liquid formulation. Doses may be necessary at various frequencies as well. For instance, in some embodiments, a subject may be administered a one dose per day, two doses per day (e.g., spaced approximately 12 hours apart), three doses per day, four doses per day, five doses per day, or simply as needed. In some embodiments, daily dosing may not be required, and a subject may be administered one or more doses every other day, every three days, every four days, every five days, every six day, or every week.

Without being bound by theory, it is believed that the disclosed nanoemulsion formulation comprising at least one cannabinoid or a salt or derivative thereof treat uncomfortable arthritis conditions through a combination of analgesic and anti-inflammatory effects. The cannabinoids or a salt or derivative thereof may also decrease ocular pressure. In embodiments further comprising a second active compound, such as Diclofenac sodium or a salt or derivative thereof, additional synergistic benefit is expected because the cannabinoids and Diclofenac sodium function via different mechanisms and effect different pathways. As a result, the combination of at least one cannabinoid or a salt or derivative thereof and Diclofenac sodium or a salt or derivative thereof will be more efficacious than administration of Diclofenac sodium alone, produce fewer side effects (e.g., irritation and stomach ulcers), and result in a more rapid onset of the intended therapeutic effect of eliminating, reducing, or ameliorating at least one or more signs or symptoms of joint pain. Indeed, treating joint pain with the novel combination of Diclofenac sodium or a salt or derivative thereof and at least one cannabinoid or a salt or derivative thereof is expected to provide numerous clinical benefits, as discussed herein, regardless of the precise formulation used.

A person of ordinary skill in the art would understand that the degree of skin irritation that a given arthritis treatment produces can be determined via, for example, acute skin irritation tests in animals. These tests may include, for example, pretreatment with an appropriate anesthetic followed by application of the experimental treatment (i.e., one of the disclosed nanoemulsion formulations or a Diclofenac sodium formulation without a cannabinoid) to the skin of an experimental animal, such as a rabbit, rat, or mouse. Untreated animals may serve as a control. The degree of skin irritation may be evaluated by scoring lesions of the skin at time intervals. The duration of the study may be long enough to determine whether any adverse effects are reversible.

Similarly, a person of ordinary skill in the art would understand that the onset of therapeutic effect can be determined through, for example, tests, as required by the U.S. FDA. Other suitable endpoints may include, but are not limited to, a decrease in pain, irritation, redness, or dryness of a individual's skin following treatment with a given therapeutic (i.e., one of the disclosed nanoemulsion formulations or a Diclofenac sodium formulation without a cannabinoid). In some embodiments, treatment with the disclosed nanoemulsion formulations or, in particular, with the combination of Diclofenac sodium or a salt or derivative thereof and at least one cannabinoid or a salt or derivative thereof may result in the onset of therapeutic activity within less than about 3 months, less than about 2 months, less than about 1 month, less than about 3 weeks, less than about 2 weeks, or less than about 1 week.

### Definitions

As used in the description of the invention, the singular forms “a”, “an” and “the” are used interchangeably and intended to include the plural forms as well and fall within each meaning, unless the context clearly indicates otherwise. Also, as used herein, “and/or” refers to and encompasses any and all possible combinations of one or more of the listed items, as well as the lack of combinations when interpreted in the alternative (“or”).

As used herein, the term “about” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term.

As used herein, the phrases “therapeutically effective amount” and “therapeutic level” mean that drug dosage that provides the specific pharmacological effect for which the drug is administered in a subject that is in ned of treatment for joint pain, i.e., to reduce, ameliorate, or eliminate the symptoms or effects of joint pain. It is emphasized that a therapeutically effective amount or therapeutic level of a drug will not always be effective in treating the conditions described herein, even though such dosage is deemed to be a therapeutically effective amount by those of skill in the art. For convenience only, exemplary dosages, drug delivery amounts, therapeutically effective amounts, and therapeutic levels are provided herein. The therapeutically effective amount may vary based on the route of administration and dosage form, the age and/or weight of the subject, and/or the subject's condition, including the severity of the condition being treated.

The terms “treatment” or “treating” as used herein with reference to joint pain or arthritis conditions mean reducing, ameliorating or eliminating one or more symptoms or effects of joint pain, which may include, but are not limited to, pain, irritation, redness, and, dryness.

The terms “individual,” “subject,” and “patient” are used interchangeably herein, and refer to any individual mammal subject, e.g., bovine, canine, feline, equine, or human. In preferred embodiments, the subject is a human.

The terms “administration of” or “administering” an active agent or nanoemulsion of the present disclosure should be understood to mean providing the agent or nanoemulsion to a subject in need of treatment by a means that can be introduced into that individual's body in a therapeutically useful form and therapeutically effective amount. For example, the disclosed nanoemulsions may be administered topically, i.e., directly on the skin of the subject.

The term “nanoemulsion” as used herein, includes small oil-in-water dispersions or droplets, as well as other lipid structures which can form as a result of hydrophobic forces which drive apolar residues (e.g., long hydrocarbon chains) away from water and drive polar head groups toward water, when a water immiscible oily phase is mixed with an aqueous phase. These other lipid structures include, but are not limited to, unilamellar, paucilamellar, and multilamellar lipid vesicles, micelles, and lamellar phases. The present disclosure contemplates that one skilled in the art will appreciate this distinction when necessary for understanding the specific embodiments herein disclosed. Nanoemulsion particle size may vary, but lipid droplets in the emulsion will tend to be less than about 1 micron, or more specifically, less than about 500, about 400, about 300, about 200, about 175, about 150, or about 125 nm in diameter (or other particles sizes described herein).

The term “multiphasic” as used in the context of the present disclosure means that an active agent (e.g., THC, CBD, CBDi and/or Diclofenac sodium) can be found in multiple phases of a given nanoemulsion. For example, a given nanoformulation may be “triphasic,” meaning the active agent is dissolved in the aqueous phase and oil phase of the nanoemulsion, as well as being in a particulate form in suspension within the nanoemulsion. The unique multiphasic nature of the disclosed nanoemulsion formulations may be attributed to the high pressure (i.e., >10,000 psi) homogenization process used to produce the nanoemulsions.

As used herein, the term “isolated” as used in the present disclosure refers to cannabinoids or salts or derivatives thereof that are independent from their natural location. In other words, isolated cannabinoids may be directly obtained from a cannabis plant or synthesized.

The disclosed nanoemulsion formulations and methods of using the same are further described below by reference to the examples, which are provided for illustration only. The invention is not limited to the examples, but rather includes all variations that are evident from the teachings provided herein. All publicly available documents referenced herein, including but not limited to U.S. patents, are specifically incorporated by reference.

### EXAMPLES

### Example 1—Treatment of Joint Pain in a Human Subject

This example describes the use of the disclosed Diclofenac sodium and cannabinoid-comprising nanoemulsions in the treatment of an arthritis condition such as joint pain in a subject. In this example, at least one cannabinoid (e.g., CBD or CBDi) is formulated in the disclosed nanoemulsion with Diclofenac sodium for the purposes of preparing a pharmaceutical product.

Subjects suspected of having or diagnosed with joint pain receive at least one administration of a therapeutically effective amount of a disclosed nanoemulsion, either alone or in combination with one or more additional agents for the treatment or prevention of joint pain. The disclosed nanoemulsion (and optional additional agents) are administered directly on the skin according to methods known in the art. Subjects will be evaluated regularly for the presence and/or severity of signs and symptoms associated with joint pain, including, but not limited to, e.g., dry, irritated or red skin, irritation, and/or pain. Treatments may be maintained until such a time as one or more signs or symptoms of joint pain are prevented.

It is believed that subjects receiving a therapeutically effective amount of a disclosed cannabinoid-containing Diclofenac sodium nanoemulsion, will have a reduced or abolished signs or symptoms of joint pain, and that these subjects will experience a more rapid clinical improvement than subjects receiving a comparable dose of Diclofenac sodium without a cannabinoid. It is further expected that administration of the disclosed nanoemulsions in combination with one or more additional agents will have additive or synergistic effects in this regard.

These results will show that disclosed nanoemulsions are useful in the prevention and treatment of arthritis conditions such as joint pain.

### Example 2—Nanoemulsions of the Invention

The examples below illustrate diclofenac sodium and cannabinoid nanoemulsions.

Multiple test batches of nanoemulsions have been prepared and analyzed. The tables below provides the components of several of the exemplary nanoemulsion formulations that have been prepared.

As the following will show, only certain formulations having long-term stability worked, while

formulations using Coconut Oils and using more than 2% trancutol-P failed. Formulations using Brand B oils as well as Brand C oils failed.

The following relates to batches of (1) DSS (Diclofenac sodium) formulations (2) DSS-CBD formulations that were deemed successful:

For stability studies for the formulations described in the specification, samples were kept in (1) accelerated chamber conditions, the accelerated chamber conditions being at 40 degrees Centigrade and 75% relative humidity (ACC) and (3) refrigeration. As per FDA guidelines, one month ACC stability is equivalent to roughly four months stability at room temperature, room temperature (RT) of 25 degrees Centigrade. As of the time of the filing of the specification, samples that failed ACC conditions are still being observed at room temperature and refrigeration. For many of the examples provided below, samples analysis is in progress. All of the samples were packed in amber glass bottles for stability studies.

The following relates to examples of (1) DSS formulations; (2) DSS-CBDi formulations; and (3) DSS-CBDi-THC formulations. The formulations with 6 months STB are successful formulations. STB is Stability condition which is 40° C. and 75% RH.

TABLE 1
DSS only batches

| | DSS (1) | DSS (1) | DSS (1) | DSS (1) | DSS (1) | DSS(1) | DSS (1) | DSS (1) | DSS (1) |
|---|---|---|---|---|---|---|---|---|---|
| | #1 (6 | #2 (6 | #3 (6 | #4 (5 | #5 (0 | #6 (4 | #7 (3 | #8 (3 | #9 (3 |
| | MONTHS | MONTHS | MONTHS | MONTHS | MONTHS | MONTHS | MONTHS | MONTHS | MONTHS |
| Ingredients | STB) | STB) | STB) | STB) | STB) | STB) | STB) | STB) | STB) |
| Result: | Success | Success | Success | Failure | Failure | Failure | Failure | Failure | Failure |
| DSS | 1 | 1 | 1 | 1 | 1 | 1 | 1 | 1 | 1 |
| Brand C | 38.5 | — | — | — | — | — | — | 38 | 38 |
| Sesame Oil | | | | | | | | | |
| Brand A | — | 38.25 | — | — | — | — | — | — | — |
| Safflower Oil | | | | | | | | | |
| Brand D | — | — | 38.25 | 35.75 | — | — | — | — | — |
| Olive Oil | | | | | | | | | |
| MCT | — | — | — | — | 38.25 | — | — | — | — |
| Coconut Oil | | | | | | | | | |
| Brand A | — | — | — | — | — | 40.25 | 38 | — | — |
| Medium Chain | | | | | | | | | |
| Triglycerides | | | | | | | | | |
| Ethanol | 9 | 9 | 9 | 13 | 9 | 8 | 9 | 13 | 13 |
| Poly-80 | 14 | 14 | 14 | 14 | 14 | 6 | 7 | 8 | 8 |
| Water | 37.25 | 37.25 | 37.25 | 35.75 | 37.25 | 41.75 | 40 | 36.6 | 36.9 |
| Alpha-toco | 0.5 | 0.5 | 0.5 | | 0.5 | 0.5 | — | — | — |
| SEGT | — | — | — | — | — | 2.5 | 3.5 | 3 | 3 |
| Propylene | — | — | — | — | — | — | 1.5 | — | — |
| Glycol | | | | | | | | | |
| BCD | — | — | — | — | — | — | — | 0.4 | 0.1 |

From the foregoing Table 1, some observations can be made. The abbreviation “DSS” refers to DSS is Diclofenac Sodium Salt. The described formulations are applicable for dermal applications, for example. The following describes relevant points about the formulations.

Stable formulations had a stability of six months under STB and had no phase separation.

DSS (1) #1 had a stability of six months while containing 38.5% of Brand C sesame oil and 37.25% water.

DSS (1) #2 had a stability of six months while containing 38.25% of Brand A safflower oil and 37.25% water.

DSS (1) #3 had a stability of six months while containing 38.25% of Brand D olive oil and 37.25% water.

From a review of the data from successful examples 1-3, critical factors for stability included the following factors: (1) a water concentration range of about 35-38%, (2) an oil concentration range of about 35 to 38%; (3) a pH range of 7-8.3, as none of the formulations had phase separation.

DSS (1) #4 failed at five months as it contained an oil concentration of about 35 percent, indicating that a greater oil concentration of about 38 percentage is more preferred for a greater stability in this particular formulation.

DSS (1) #5 failed and separated into two phases, an oil phase and a water phase for this particular addition of coconut oil.

DSS formulations #6-9 may have lower stability due to the presence of SEGT, PG and BCD and accordingly failed as each of the oils did not have oil concentration in the mentioned critical concentrations.

Formulation #6 and #7 contain Brand A oil. Formulation #8 and Formulation #9 contain Brand C Sesame Oil.

Brand A Oil is Medium chain triglyceride derived from coconut or palm kernel oil. January 26^{th} note: Brand B may be Brand B sesame oil or Brand B olive oil; Brand C sesame is a sesame oil which is large chain (about 50-710) triglycerides.

A person of ordinary skill in the article can utilize the respective amounts from each of the successful formulations #1-3 and apply the following procedure to create the respective formulation.

in a known quantity of Ethanol, sonication is applied until a clear layer is formed. To the foregoing resulting solution, alpha-toco and a desired oil (for example, in formulation #1, Brand C Sesame Oil, in formulation #2, Brand A Safflower Oil, and so on) was added and sonicated again till a clear layer is formed.

After that, Poly-80 was added to the resulting solution and sonicated again till a clear single homogenous layer is formed. A subsequent oil phase developed. In another beaker, a known quantity of water was measured and added to the oil phase while mixing by using an electrical stirrer at around 1200 rpm for about 15 minutes. After stirring, the pH was measured for the formulation and it was found to around 8. The emulsion was passed through the homogenizer for three cycles at 10,000-15,000 psi and a final formulation was formed.

The following is Table 2, which shows the failure of a cannabinoid containing DSS formulation having THC, despite including an oil successfully used in a co-pending application filed by Applicant.

TABLE 2
DSS + CBDi + THC batches

| | DSS(1) #10 | DSS(1) #11 | DSS(1) #12 | DSS(1) #13 |
|---|---|---|---|---|
| Ingredients | (0M STB) | (0M STB) | (0M STB) | (0M STB) |
| DSS | 1 | 1 | 1 | 1 |
| CBDi | 3 | 3 | 3 | 3 |
| THC | 1 | 1 | 1 | 1 |
| Brand A | | 12 | 22 | — |
| Safflower Oil | | | | |
| Brand A | 37.3 | — | — | — |
| Olive Oil | | | | |
| Brand A | — | — | — | 33.02 |
| Medium Chain | | | | |
| Triglyceride | | | | |
| Ethanol | 9 | 9 | 9 | 7.27 |
| Poly-80 | 8 | 10 | 6 | 6.08 |
| Water | 40.5 | 47.5 | 34 | 40.5 |
| Alpha-toco | — | 0.3 | — | — |
| SEGT | — | — | — | 3.04 |
| MP Propylene | — | 1.5 | 1.5 | 0.93 |
| Glycol | | | | |
| Brand D Proply | 0.2 | 0.2 | — | — |
| Gallate | | | | |
| Brand A | — | 14.5 | 22.5 | 4.16 |
| Oleic Acid | | | | |

In contrast to Table 1, the DSS formulations in Table 2 additionally included addition of cannabinoids, CBDi and THC. The addition of THC led to failure and none of the formulations had stability. The foregoing formulations in Table 2 are intended for dermal applications.

Co-pending U.S. application Ser. No. 17/401,826 filed on Aug. 13, 2021 by Applicant disclosed the criticality of using Brand A oil in cannabinoid-alone formulations and cannabinoid formulations containing cyclosporine. Brand A oil is a pharmaceutically acceptable oil having at least one oxidizable metal of less than one part per million in said oil, wherein the at least one oxidizable metal is one or more of the following: a copper content of less than one part per million in said oil; a chromium content of less than one part per million in said oil; a lithium content of less than one part per million in said oil; and a molybdenum content of less than one part per million in said oil.

Despite including a Brand A oil in a DSS formulation containing THC, the formulations failed. This showed that inclusion of a Brand A oil, although critical for cannabinoid-alone formulations and cannabinoid formulations containing cyclosporine, is not a factor for stability of a cannabinoid-containing DSS formulation having THC. Thus, it would not be obvious for a person of ordinary skill to modify the formulation as disclosed in U.S. application Ser. No. 17/401,826 and its teachings to yield a cannabinoid containing DSS formulation having THC.

The following is Table 3, which describes formulations intended to be used for ophthalmic use.

TABLE 3
0.1% DSS + CBDi + THC batches

| | DSS(0.1) | DSS(0.1) | DSS(0.1) | DSS(0.1) | DSS(0.1) | DSS(0.1) | DSS(0.1) |
|---|---|---|---|---|---|---|---|
| | (5 M RT) | (6 M RT) | (8 M RT) | (4 M RT) | (4 M RT) | (4 M RT) | (4 M RT) |
| Ingredients | #14 | #15 | #16 | #17 | #18 | #19 | #20 |
| Result | Failure | Success | Success | Failure | Failure | Failure | Failure |
| DSS | 0.1 | 0.1 | 0.1 | 0.1 | 0.1 | 0.1 | 0.1 |
| CBDi | 1 | 1 | 1 | 1 | 1 | — | — |
| THC | 2 | 2 | 2 | 2.01 | 2 | — | — |
| Brand A Sesame | — | 2 | 1 | 1 | — | — | — |
| Oil | | | | | | | |
| Brand A Safflower | — | — | 1 | — | — | — | — |
| Oil | | | | | | | |
| Brand A Medium | 6.55 | 4.55 | 4.55 | 6.55 | 6.55 | 6.55 | 6.55 |
| Chain Triglyceride | | | | | | | |
| Ethanol | 0.1 | 0.2 | 0.3 | 0.1 | 0.1 | 0.2 | 0.2 |
| Poly-80 | 2 | 2 | 2 | 2 | 2 | 2 | 2 |
| Povidone | — | — | — | — | — | 0.1 | — |
| SEGT | — | — | — | 0.01 | — | — | — |
| Sodium Hyaluronate | — | — | — | — | 0.01 | — | 0.1 |
| BCD | 0.01 | 0.01 | 0.01 | — | 0.01 | 0.01 | 0.01 |
| MPBS | 88.24 | 88.14 | 88.04 | 88.22 | 88.23 | 91.04 | 91.04 |

Stable formulations had a stability of six months under room temperature and had no phase separation.

The successful formulation included Formulations #15 and 16, which contained a Brand A oil being a pharmaceutically acceptable oil having at least one oxidizable metal of less than one part per million in said oil, wherein the at least one oxidizable metal is one or more of the following: a copper content of less than one part per million in said oil; a chromium content of less than one part per million in said oil; a lithium content of less than one part per million in said oil; and a molybdenum content of less than one part per million in said oil. Besides Brand A oil, other oils may be used.

With respect to DSS formulation #16, addition of Brand A safflower oil led to increased stability, with a stability of 8 months.

The following criteria are believed to be necessary for a successful formulation: (1) 4-10% oil with an exemplary preferred range of 4-5%; (2) an ethanol concentration of at least 0.2 to 0.3 percentage was critical; (3) 90-95% water; (4) pH of 7 to 8.0.

Co-pending U.S. application Ser. No. 17/401,826 filed on Aug. 13, 2021 by Applicant disclosed the criticality of using Brand A oil in cannabinoid-alone formulations and cannabinoid formulations containing cyclosporine, as mentioned in Table 2. However, with this formulation, a person of ordinary skill could utilize oils other than Brand A oil.

However, the mentioned co-pending application additionally failed to disclose other critical factors necessary to create an ophthalmic cannabinoid containing DSS formulation having THC, such as ethanol concentration and a critical oil percentage.

pH is an important critical factor for these formulations unlike cyclosporine, which is not a salt.

Hence it would not be obvious for a person of ordinary skill to modify the formulation as disclosed in U.S. application Ser. No. 17/401,826 and its teachings to yield a cannabinoid-containing DSS formulation having THC.

Notably, the concentration of MPBS does not appear to be a factor for imparting stability for a cannabinoid containing DSS formulation having THC. MPBS is a combination of PBS Buffer: Water (50:50). So, changing the concentration of MPBS from about 88%-91% and keeping the concentration of oil in the range of about 7% does not affect the stability of the formulation #14 to #20.

### Experimental Procedure for Formulation #15

A person of ordinary skill in the article can utilize the respective amounts from each of the successful formulations #15-16 and apply the following procedure to create the respective formulation.

In a Beaker, DSS, CBDi and THC were weighed and ethanol was added to it and sonicated till dissolving was complete and a single layer was formed. Brand A MCT oil and Sesame oil were added to it and sonicated again till a clear single layer was formed.

After that, Brand A poly-80 was added to the resulting layer and sonicated till a clear single layer was formed again. Thus, an oil phase was created. In another beaker, known quantity of BCD was weighed and dissolved in known quantity of MPBS. This solution was added to the oil phase while mixing using an electrical stirrer at around 1200 rpm for about 15 minutes. After stirring, the pH was measured, and it was found to around 7. The emulsion was passed through the homogenizer for 3 cycles at 10,000-15,000 psi.

Although Brand A oil was utilized, other oils with the mentioned percentage can be used.

The following table illustrates examples of a dermal cannabinoid containing DSS formulation having CBDi.

TABLE 4
1% DSS + CBDi batches

| | DSS(1) | DSS(1) | DSS(1) | DSS(1) | DSS(1) | DSS(1) | DSS(1) | DSS(1) | DSS(1) | DSS(1) |
|---|---|---|---|---|---|---|---|---|---|---|
| | (0 M | (0 M | #23 | #24 | #25 | #26 | #27 | #28 | #29 | #30 |
| | STB) | STB) | 6 M | 6 M | 6 M | 6 M | 6 M | 7 M | 6 M | 6 M |
| Ingredients | #21 | #22 | STB | STB | STB | STB | STB | STB | STB | STB |
| Result | Failure | Failure | Success | Success | Success | Success | Success | Success | Success | Success |
| DSS | 1 | 1 | 1 | 1 | 1 | 1 | 1 | 1 | 1 | 1 |
| CBDi | | 5 | | | | | | | | |
| Brand A | — | — | 33 | 44 | 38 | 36 | 37 | 36.25 | 40 | 38.15 |
| Safflower Oil | | | | | | | | | | |
| Brand B | 36.25 | 34.025 | — | — | — | — | — | — | — | — |
| Lipohile | | | | | | | | | | |
| Labrafil | 12 | 14 | — | — | — | — | — | — | — | — |
| M1944 | | | | | | | | | | |
| Transcutol-P | — | 2 | — | — | — | — | — | — | — | — |
| Ethanol | 11 | 9 | 10 | 10 | 9 | 10 | 10 | 9 | 10 | 9 |
| Poly-80 | — | — | 9 | 9 | 6 | 9 | 9 | 14 | 9 | 13 |
| Water | 35.25 | 34.275 | 47 | 37 | 37 | 44 | 43 | 37.25 | 40 | 37.15 |
| Alpha-toco | 0.5 | 0.5 | — | — | — | — | — | 0.5 | — | 0.5 |
| EPTS | — | — | — | — | — | — | — | 2 | — | — |
| SEGT | 4 | — | — | — | 3.5 | — | — | — | — | — |
| MP Propylene | — | — | — | — | 5 | — | — | — | — | — |
| Glycol | | | | | | | | | | |
| Brand D | — | 0.2 | — | — | — | — | — | — | — | — |
| Proply Gallate | | | | | | | | | | |
| Brand A | — | — | — | — | 0.5 | — | — | — | — | 1 |
| Oleic Acid | | | | | | | | | | |
| BCD | — | — | — | — | — | — | — | — | — | 0.2 |

Stable formulations had a stability of six months under STB conditions and had no phase separation.

From a review of the successful formulations, the following criteria are paramount factors for successful formulations: (1) an oil ratio of 33-40%; (2) a water concentration of from 37.25% to 47%; and (3) a pH range of 7.8-8.4. Although Brand A oil was used, oils other than Brand A can be used.

Both formulations #21 and #22 failed. It has separated into oil and water phases in less than a month.

A person of ordinary skill in the article can utilize the respective amounts from each of the mentioned successful formulations. Procedures similar to those described in Tables 2 and 3 may be utilized, although with some modifications, since the formulation is directed to 1% DSS+CBDi batches.

The same discussion concerning inapplicability of Co-pending U.S. application Ser. No. 17/401,826 relating to Table 3 may be applicable for Table 4.

The following additional batches demonstrate the effect on various formulation changes on stability.

The following batches (1 through 7) demonstrate that the addition of glycerin led to failure.

TABLE 5
DSS + CBDi (or Ci)

| | | DSS + Ci | DSS + Ci | DSS + Ci | DSS + Ci | DSS + Ci | DSS + Ci |
|---|---|---|---|---|---|---|---|
| | DSS + Ci | #2 | #3 | #4 | #5 | #6 | #7 |
| | #1 | 4RT + | 4 RT + | 5 RT + | 5 RT + | 4.5 RT + | 5 RT + |
| Ingredients | 3 M ACC | 3 M ACC | 2 M ACC | 2 M ACC | 2 M ACC | 2 M ACC | 3 M ACC |
| Result | STABLE | STABLE | STABLE | FAILED | FAILED | FAILED | STABLE |
| | | | | | (ACC | | |
| | | | | | SEPARATED) | | |
| DSS | 2 | 2 | 2 | 2 | 2 | 2 | 2 |
| CBDi | 10 | 10 | 10 | 10 | 10 | 10 | 10 |
| REFINED | 36 | 33 | 34 | 34 | 34 | 31 | — |
| OLIVE OIL | | | | | | | |
| SAFFLOWER | | | | | | | 36 |
| OIL | | | | | | | |
| Brand B | — | — | — | — | — | — | — |
| Lipohile | | | | | | | |
| CEA | 1.5 | 1.5 | 1.5 | 1.5 | 1.5 | 1.5 | 1.5 |
| Labrafil | — | — | — | — | — | — | — |
| M1944 | | | | | | | |
| Transcutol-P | — | — | — | — | — | — | — |
| Ethanol | | — | — | — | — | — | — |
| ACROS Poly-80 | 4 | 7 | 6 | 6 | 6 | 7 | 4 |
| MILLIPORE | 45 | 45 | 45 | 43 | 40 | 42 | 45 |
| Water | | | | | | | |
| GLYCERIN | | | | 2 | 5 | 5 | |
| Alpha-toco | — | — | — | — | — | — | — |
| EPTS | — | — | — | — | — | — | — |
| SEGT | — | — | — | — | — | — | — |
| CARBOPOL | 1.5 | 1.5 | 1.5 | 1.5 | 1.5 | 1.5 | 1.5 |
| MP Propylene | — | — | — | — | — | — | — |
| Glycol | | | | | | | |

In The following batches (8 through 15), batch 11 failed because of the addition of Tween 80.

TABLE 6
DSS + CBDi (or Ci)

| | | DSS + Ci | | | | | | |
|---|---|---|---|---|---|---|---|---|
| | DSS + Ci | #9 | | DSS + Ci | DSS + Ci | DSS + Ci | DSS + Ci | DSS + Ci |
| | #8 | 5RT + | DSS + Ci | #11 | #12 | #13 | #14 | #15 |
| | 5RT + | 0 M ACC | #10 | 5 RT + | 5 RT + | 4 RT + | 2 RT + | 4 RT + |
| Ingredients | 5 M ACC | (SEPARATED) | 3 M ACC | 0 M ACC | 2 M ACC | 3 M ACC | 2 M ACC | 3 M ACC |
| Result | STABLE | FAILED | STABLE | FAILED | FAILED | STABLE | FAILED | STABLE |
| DSS | 2 | 2 | 2 | 2 | 2 | 2 | 2 | 2 |
| CBDi | 10 | 10 | 10 | 10 | 10 | 10 | 10 | 10 |
| REFINED | — | — | 36 | — | — | — | — | — |
| OLIVE OIL | | | | | | | | |
| SESAME OIL | | | | | 36 | 36 | 36 | 36 |
| CR.GTCC OIL | | | | 33 | | | | |
| SAFFLOWER OIL | | 36 | | | | | | |
| SOYABEAN OIL | 36 | | | | | | | |
| TCI TWEEN 80 | | | | 4 | | | | |
| Brand B | — | — | — | — | — | — | — | — |
| Lipohile | | | | | | | | |
| CEA | 1.5 | 1.5 | 1.5 | 5 | 1.5 | 1.5 | 1.5 | 1.5 |
| Labrafil | — | — | — | — | — | — | — | — |
| M1944 | | | | | | | | |
| Transcutol-P | — | — | — | — | — | — | — | — |
| Ethanol | | — | — | — | — | — | — | — |
| ACROS Poly-80 | 4 | 4 | 4 | — | 4 | 4 | 4 | 4 |
| MILLIPORE | 45 | 45 | 45 | 46 | 45 | 45 | 45 | 45 |
| Water | | | | | | | | |
| GLYCERIN | | | | | — | — | — | — |
| Na Benzoate | | | | | | | | |
| Alpha-toco | — | — | — | — | — | — | — | — |
| EPTS | — | — | — | — | — | — | — | — |
| SEGT | — | — | — | — | — | — | — | — |
| CARBOPOL | 1.5 | 1.5 | 1.5 | 1.5 | 1.5 | 1.5 | 1.5 | 1.5 |
| MP Propylene | — | — | — | — | — | — | — | — |
| Glycol | | | | | | | | |

In the following batches (16 through 23), batch 23 failed because of the excess amount of arlasolve oil and batch 18 failed because sodium benzoate was added without transcutol-p.

TABLE 7
DSS + CBDi (or Ci)

| | DSS + Ci | DSS + Ci | DSS + Ci | DSS + Ci | DSS + Ci | DSS + Ci | DSS + Ci | DSS + Ci |
|---|---|---|---|---|---|---|---|---|
| | #16 | #17 | #18 | #19 | #20 | #21 | #22 | #23 |
| | 6RT + 1 | 5 RT + 2.5 | 6RT + 2 | 6RT + 0 | 6 RT + 4 | 2RT + 0 | 6 RT + 6 | 6RT + 0 |
| Ingredients | M ACC | M ACC | M ACC | M ACC | M ACC | M ACC | MACC | M ACC |
| Result | FAILED | FAILED | FAILED | FAILED | STABLE | FAILED ACC | STABLE | FAILED ACC |
| | | | | | | SEPARATED | | SEPARATED |
| | | | | | | BEFORE | | BEFORE |
| | | | | | | ANALYSIS | | ANALYSIS |
| DSS | 2 | 2 | 2 | 3 | 2 | 3 | 2 | 2 |
| CBDi | 10 | 10 | 10 | 10 | 10 | 10 | 10 | 10 |
| REFINED | — | — | — | — | — | — | — | — |
| OLIVE OIL | | | | | | | | |
| SESAME OIL | 34 | 35 | 35.9 | 37 | 34 | 36 | — | |
| CR.GTCC OIL | | | | | | | 35 | |
| SAFFLOWER OIL | | | | | | | | |
| SOYABEAN OIL | | | | | | | | |
| TCI TWEEN 80 | | | | | | | | |
| Brand B Lipohile | — | — | — | — | — | — | — | — |
| CEA | 1.5 | 1.5 | — | 3 | 2 | 3 | 1.5 | 1.5 |
| ARLASOLVE OIL | | | | | | | 3 | 36 |
| Labrafil M1944 | — | — | — | — | — | — | — | — |
| Transcutol-P | — | — | — | — | 2 | — | — | |
| Ethanol | — | — | 1.5 | — | — | — | — | — |
| ACROS Poly-80 | 4 | 4 | 4 | 7 | 5 | 10 | 6 | 4 |
| MILLIPORE Water | 42 | 44 | 44.9 | 37.9 | 43 | 36 | 41 | 45 |
| GLYCERIN | 5 | 2 | | — | — | — | — | |
| Na Benzoate | | | 0.1 | 0.1 | 0.1 | 0.1 | | |
| POTASSIUM | | | 0.1 | | | | | |
| SORBATE | | | | | | | | |
| Alpha-toco | — | — | — | — | — | — | — | — |
| EPTS | — | — | — | — | — | — | — | — |
| SEGT | — | — | — | — | — | — | — | — |
| CARBOPOL | 1.5 | 1.5 | 1.5 | 2 | 2 | 2 | 1.5 | 1.5 |
| MP Propylene | — | — | — | — | — | — | — | — |
| Glycol | | | | | | | | |

The following batches (24 through 31) demonstrate that the addition of excess arlasolve oil, the addition of SEGT and the addition of transcutol-p without sodium benzoate led to failure.

TABLE 8
DSS + CBDi (or Ci)

| | DSS + Ci | DSS + Ci | DSS + Ci | DSS + Ci | DSS + Ci | DSS + Ci | DSS + Ci | DSS + Ci |
|---|---|---|---|---|---|---|---|---|
| | #24 | #25 | #26 | #27 | #28 | #29 | #30 | #31 |
| | 6RT + 1 | 5RT + 0 | 6 RT + 3.5 | 6 RT + 6 | 6 RT + 3.5 | 8 RT + 4 | 5 RT + 5 | 6RT + 2 |
| Ingredients | M ACC | M ACC | M ACC | M ACC | M ACC | M ACC | M ACC | M ACC |
| Result | FAILED ACC | FAILED ACC | STABLE | STABL | STABL | STABLE | STABLE | FAILED |
| | SEPARATED | SEPARATED | ACC | | | | | |
| | BEFORE | BEFORE | SEPARATED | | | | | |
| | ANALYSIS | ANALYSIS | AFTER 3 M | | | | | |
| DSS | 2.5 | 2 | 1.5 | 1.5 | 1.5 | 1.5 | 1.5 | 1.5 |
| CBDi | 10 | 7.5 | 7.5 | 10 | 9 | 9 | 8 | 8 |
| REFINED | — | | — | — | — | — | — | — |
| OLIVE OIL | | | | | | | | |
| SESAME OIL | | | 37 | 35.5 | | 36.5 | | 36.5 |
| CR.GTCC OIL | | 31.76 | | | | | | |
| SAFFLOWER OIL | | | | | 36.5 | | 36.5 | |
| SOYABEAN OIL | | | | | | | | |
| ARLASOLVE OIL | 34 | | | | | | | |
| TCI TWEEN 80 | | | | | | | | |
| Brand B Lipohile | — | — | — | — | — | — | — | — |
| CEA | 1.5 | — | 5 | 1.5 | 1.5 | 1.5 | 1.5 | 1.5 |
| Labrafil M1944 | — | — | — | — | — | — | — | — |
| Transcutol-P | — | — | — | — | — | — | — | 1 |
| Ethanol | — | 12 | — | — | — | — | — | — |
| ACROS Poly-80 | 5 | 6 | 4 | 4 | 4 | 4 | 4 | 4 |
| MILLIPORE | 45.5 | 38.74 | 47 | 46 | 46 | 46 | 47 | 47 |
| Water | | | | | | | | |
| GLYCERIN | | | | — | — | — | — | |
| Na Benzoate | | | | | | | | |
| Alpha-toco | — | — | — | — | — | — | — | — |
| EPTS | — | — | — | — | — | — | — | — |
| SEGT | — | 2 | — | — | — | — | — | — |
| CARBOPOL | 1.5 | | 1.5 | 1.5 | 1.5 | 1.5 | 1.5 | 1.5 |
| MP Propylene | — | — | — | — | — | — | — | — |
| Glycol | | | | | | | | |

The following batches (32 through 38) demonstrate that the addition of castor oil led to failure.

TABLE 9
DSS + CBDi (or Ci)

| | DSS + Ci | DSS + Ci | DSS + Ci | DSS + Ci | DSS + Ci | DSS + Ci | DSS + Ci |
|---|---|---|---|---|---|---|---|
| | #32 | #33 | #34 | #35 | #36 | #37 | #38 |
| | 6RT + 3 | 5RT + 3 | 6RT + 1 | 6 RT + 5 | 6 RT + 0 | 6RT + 4 | 8.5RT + 5.5 |
| Ingredients | M ACC | M ACC | M ACC | M ACC | M ACC | M ACC | M ACC |
| Result | STABLE | STABLE | FAILED ACC | STABLE | FAILED ACC | STABLE | STABLE |
| | | | SEPARATED | | SEPARATED | | |
| | | | AFTER 1 M | | BEFORE | | |
| | | | | | ANALYSIS | | |
| DSS | 1.5 | 2 | 1.75 | 1.75 | 1.5 | 1.5 | 2 |
| CBDi | 7.5 | 9 | 9 | 9 | 9 | 7.5 | 10 |
| REFINED | — | — | | — | — | — | 36 |
| OLIVE OIL | | | | | | | |
| SESAME OIL | | | | — | — | | |
| CR.GTCC OIL | | 36.5 | | 36.25 | | 37 | |
| SAFFLOWER OIL | 37 | | | | | | |
| CASTOR OIL | | | 36.25 | | 36.5 | | |
| SOYABEAN OIL | | | | | | | |
| ARLASOLVE OIL | | | | | | | |
| TCI TWEEN 80 | | | | | | | |
| Brand B Lipohile | — | — | — | — | — | — | — |
| CEA | 1.5 | 1.5 | — | 1.5 | 1.5 | 1.5 | — |
| Labrafil M1944 | — | — | — | — | — | — | — |
| Transcutol-P | — | — | — | — | — | — | — |
| Ethanol | | — | — | — | — | — | 1.5 |
| ACROS Poly-80 | 4 | 4 | 4 | 4 | 4 | 4 | 4 |
| MILLIPORE | 47 | 45.5 | 46 | 46 | 46 | 47 | 45 |
| Water | | | | | | | |
| GLYCERIN | | | | | — | — | |
| Na Benzoate | | | | | | | |
| Alpha-toco | — | — | — | — | — | — | — |
| EPTS | — | — | — | — | — | — | — |
| SEGT | — | — | — | — | — | — | — |
| CARBOPOL | 1.5 | 1.5 | 1.5 | 1.5 | 1.5 | 1.5 | 1.5 |
| MP Propylene | — | — | — | — | — | — | — |
| Glycol | | | | | | | |

The following batches (39 through 44) demonstrate that the addition of Telfose 63, BHT, Labrafil, POLY 20 and excess Carbopol led to failure.

TABLE 10
DSS + CBDi (or Ci)

| | DSS + Ci | DSS + Ci | DSS + Ci | DSS + Ci | DSS + Ci | DSS + Ci |
|---|---|---|---|---|---|---|
| | #39 | #40 | #41 | #42 | #43 | #44 |
| | 8.5RT + 1 | 8.5RT + 3 | 5.5RT + 4 | 9 RT+ 1 | 6RT + 0 | 9RT + 3 |
| Ingredients | M ACC | M ACC | M ACC | M ACC | M ACC | M ACC |
| Result | FAILED ACC | STABLE | STABLE | FAILED ACC | FAILED | STABLE |
| | SEPARATED | | | SEPARATED | | |
| | AFTER 1 M | | | AFTER 1 M | | |
| DSS | 2 | 2 | 2 | 2 | 1 | 2 |
| CBDi | 10 | 10 | 10 | 10 | 5 | 10 |
| REFINED | — | — | | — | — | — |
| OLIVE OIL | | | | | | |
| SESAME OIL | | | | — | — | 36 |
| CR.GTCC OIL | | 33 | | 36 | | |
| SAFFLOWER OIL | — | | | | 35 | |
| TEFOSE 63 | 9.5 | | | | | |
| CASTOR OIL | | | 36 | | | |
| SOYABEAN OIL | | | | | | |
| ARLASOLVE OIL | | | | | | |
| TWEEN 80 | | 4 | | | | |
| GeLEOL | 2 | | | | | |
| LABRAFAC Lipohile | 4 | — | — | — | — | — |
| CEA | | — | — | | | — |
| PROPYL | | | 1 | | | |
| GALLATE | | | | | | |
| BHT | 0.1 | | | | | |
| Labrafil M1944 | 3 | — | — | — | — | — |
| Transcutol-P | — | — | — | — | — | — |
| Ethanol | | 5 | 3 | 1.5 | 6 | 1.5 |
| ACROS Poly-80 | | — | 4 | — | 3.5 | 4 |
| MILLIPORE | 64.5 | 46 | 40 | 45 | 1.5 | 45 |
| Water | | | | | | |
| POLYSORBATE 20 | | | | 4 | | |
| GLYCERIN | | | | | — | — |
| Na Benzoate | | | | | | |
| Alpha-toco | — | — | 1 | — | — | — |
| EPTS | — | — | — | — | — | — |
| SEGT | — | — | — | — | — | — |
| CARBOPOL | — | — | 2 | 1.5 | 48 | 1.5 |
| Propylene | 4.9 | — | 1 | — | — | — |
| Glycol | | | | | | |

### Combinations of Methotrexate (MTX) and DSS with Cannabidiol

Cannabidiol (CBD) is a non-psychoactive compound found in the cannabis plant and has shown promise as an analgesic, anticonvulsant, muscle relaxant, anxiolytic, antipsychotic and has shown neuroprotective, anti-inflammatory, and antioxidant activity. It is believed that when CBD is combined with DSS and MTX, this would increase the bioavailability and bio efficacy to cure RA and reducing the side effects to minimum.

The following describes two examples of a formulation comprising a combination of methotrexate, diclofenac Sodium (DSS) and cannabidiol.

TABLE 11
0.5% Methotrexate + 1% DSS + CBDi batches

| | |
|---|---|
| Methotrexate | 0.5 |
| DSS | 1 |
| CBDi | 5 |
| Ethanol | 8 |
| Alpha-Tocopherol | 0.5 |
| Brand A OIL | 37.275 |
| Propyl Gallate | 0.2 |
| BRAND A SPAN 80 | 2.5 |
| Acros Poly 80 | 6 |
| Sodium Hyaluronate | 0.1 |
| Water | 38.925 |

TABLE 12
1% Methotrexate + 1% DSS + CBDi batches

| | |
|---|---|
| Methotrexate | 1 |
| DSS | 1 |
| CBDi | 5 |
| Ethanol | 8 |
| Alpha-Tocopherol | 0.5 |
| Brand A oil | 37.025 |
| Propyl Gallate | 0.2 |
| BRAND A SPAN 80 | 2.5 |
| Acros Poly 80 | 6 |
| Sodium Hyaluronate | 0.1 |
| Water | 38.675 |

From the foregoing examples, the following are critical components of the formulation: (1) 0.5-1.0% Methotrexate; (2) 1 to 5% DSS; (3) 1 to 10% CBDI; (4) a water concentration ranging from 35 to 42 percent; (5) an oil concentration ranging from 37 percent to 63 percent; and (6) pH range of 7 to 8.2.

A person of ordinary skill in the article can utilize the respective amounts from each of the foregoing formulations and apply the following procedure to create the respective formulation.

In a beaker, Methotrexate, DSS and CBDi were weighed and dissolved in known quantity of Ethanol by sonication until a clear layer is formed. To it, Brand A GTCC oil was added and sonicated again till a clear layer is formed. After that, alpha-tocopherol, Propyl Gallate, Acros Poly-80 and BRAND A SPAN 80 was added to it and sonicated again till a clear single homogenous layer is formed. This is oil phase. In another beaker, known quantity of Sodium Hyaluronate was dissolved in water and sonicated for a while and kept in the refrigerator till its complete dissolution. This was water phase; it was added to the oil phase while mixing using an electrical stirrer at around 1200 rpm for about 15 minutes. After stirring, the pH was measured for the formulation, and it was found to around 8. The emulsion was passed through the homogenizer for 3 cycles at 10,000-15,000 psi.

### Prednisone Alone

In one example, a formulation comprising prednisone may be formulated using similar percentages of components as mentioned with DSS. Alternatively, other percentages may be contemplated. Depending on application, dermal or ophthalmic, further refinements may be made.

### Combination of Prednisone and CBDI

In one example, a formulation comprising prednisone and CBDi may be formulated using similar percentages of components as mentioned with DSS. Alternatively, other percentages may be contemplated Depending on application, dermal or ophthalmic, further refinements may be made.

### Combination of Prednisone, CBDi and THC

In one example, a formulation comprising prednisone, CBDi and THC may be formulated using similar percentages of components as mentioned with DSS. Alternatively, other percentages may be contemplated. Depending on application, dermal or ophthalmic, further refinements may be made.

### Combinations of Prednisone, Methotrexate, DSS and Cannabinoids

In one example, a formulation comprising prednisone, DSS and CBDi may be formulated using similar percentages of components as mentioned with Methotrexate. Alternatively, other percentages may be contemplated. Depending on application, dermal or ophthalmic, further refinements may be made.

In this specification, formulations using DSS or cannabinoids alone or in combination with Methotrexate or prednisone were disclosed. The concentrations of methotrexate in the pharmaceutical formulations of this invention may include about 0.5% to about 1%, about 0.5% to about 2%, about 0.5% to about 3%, about 0.5% to about 4%, about 0.5% to about 5%, about 0.5% to about 6%, about 0.5% to about 7%, about 0.5% to about 8%, about 0.5% to about 9%, or about 0.5% to about 10%. The concentrations of prednisone in the pharmaceutical formulations of this invention may include about 0.1% to about 1%, about 0.1% to about 2%, about 0.1% to about 3%, about 0.1% to about 4%, about 0.1% to about 5%, about 0.1% to about 6%, about 0.1% to about 7%, about 0.1% to about 8%, about 0.1% to about 9%, or about 0.1% to about 10%.

The teachings of the specification are not limited to formulations using the disclosed anti-inflammatory drugs. Formulations using other one or more combination of other anti-inflammatory drugs alone may be utilized. Percentages of components may be similar to components disclosed in this specification. Alternatively, other percentages may be contemplated In another example, formulations using other one or more combination of other non-steroidal anti-inflammatory drugs alone may be combined with one or more cannabinoids.

The teachings of the specification are not limited by the preferred embodiments and examples but will cover many modifications and equivalents consistent with the written description as a whole.

1. A pharmaceutical formulation comprising cannabinoids, or a salt or derivative thereof, formulated in a nanoemulsion comprising droplets having an average particle size of less than about 1 micron, wherein the cannabinoids are present in the formulation at a concentration of about 0.01 to about 15.0% (w/w).

2. A pharmaceutical formulation according to claim 1, wherein the formulation further comprises diclofenac sodium, or a salt or derivative thereof, wherein the diclofenac sodium, or a salt or derivative thereof, is present in the formulation at a concentration of about 1% to about 5% (w/w).

3. A pharmaceutical formulation comprising diclofenac sodium, or a salt or derivative thereof, formulated in a nanoemulsion comprising droplets having an average particle size of less than about 1 micron, wherein the diclofenac sodium, or a salt or derivative thereof, is present in the formulation at a concentration of about 1% to about 5% (w/w).

4. A pharmaceutical formulation according to claim 3, wherein the formulation further comprises water at a concentration of about 35 to 38% and a pharmaceutically acceptable oil at a concentration of about 35 to 38%, and further wherein the pH range of the formulation is 7 to 8.3.

5. A pharmaceutical formulation according to claim 1, wherein the formulation further comprises methotrexate, which is present in the formulation at a concentration of about 0.5% to about 1.0% (w/w).

6. A pharmaceutical formulation according to claim 1, wherein the formulation further comprises prednisone, which is present in the formulation at a concentration of about 0.1% to about 1.0% (w/w).

7. A pharmaceutical formulation according to claim 6, wherein the prednisone has a concentration of 0.1%, and the formulation further comprises 4 to 10% of a pharmaceutically acceptable oil, 0.2 to 0.3% ethanol, 90 to 95% water, and wherein the formulation has a pH range of 7.0 to 8.

8. A pharmaceutical formulation according to claim 6, wherein the prednisone has a concentration of 1.0%, and the formulation further comprises 33 to 40% of a pharmaceutically acceptable oil, 37.2 to 47% water, and wherein the formulation has a pH range of 7.8 to 8.4.

9. A pharmaceutical formulation according to claim 1, further comprising 0.1% diclofenac sodium, 4 to 10% of a pharmaceutically acceptable oil, 0.2 to 0.3% ethanol, wherein the pH of the formulation is 7 to 8.0.

10. A pharmaceutical formulation according to claim 1, wherein the cannabinoids are present in the formulation at a concentration of 1 to 10%, and wherein the formulation further comprises 0.5 to 1.0% methotrexate, 1 to 5% diclofenac sodium, water ranging from 35 to 42%, and a pharmaceutically acceptable oil ranging from 37% to 63%, wherein the pH of the formulation is in the range of 7.0 to 8.2.

11. A pharmaceutical formulation according to claim 1, wherein the nanoemulsion comprises a pharmaceutically acceptable oil having at least one oxidizable metal of less than one part per million in said oil, wherein the at least one oxidizable metal is one or more of copper, chromium, lithium, and molybdenum.

12. A pharmaceutical formulation according to claim 1, wherein the cannabinoids are present in the formulation at a concentration of about 1 to about 5% (w/w), and wherein the formulation further comprises diclofenac sodium, or a salt or derivative thereof, present in the formulation at a concentration of about 0.5% to about 2% (w/w), about 10% to about 90% (w/w) water, about 10 to about 60% (w/w) of a pharmaceutically acceptable oil, about 1 to about 15% of a surfactant, and about 0.05 to about 0.5% (w/w) of an organic solvent.

13. A pharmaceutical formulation according to claim 1, wherein the cannabinoids are present in the formulation at a concentration of about 0.01 to about 0.5% (w/w), and wherein the formulation further comprises diclofenac sodium, or a salt or derivative thereof, present in the formulation at a concentration of about 0.01% to about 2% (w/w), about 10% to about 90% (w/w) water, about 10 to about 60% (w/w) of a pharmaceutically acceptable oil, about 1 to about 15% of a surfactant, and about 0.05 to about 0.5% (w/w) of an organic solvent.

14. A pharmaceutical formulation according to claim 1, wherein the cannabinoids comprise about 0.01 to about 0.5% (w/w) tetrahydrocannabinol (THC), or a salt or derivative thereof, and about 0.01 to about 15% (w/w) cannabidiol (CBD) or CBD isolate (CBDi), or a salt or derivative thereof, wherein the formulation further comprises about 10 to about 90% (w/w) water, about 10 to about 60% (w/w) of a pharmaceutically acceptable oil, about 1 to about 15% (w/w) of a surfactant, and about 0.05 to about 0.5% (w/w) of an organic solvent.

15. A pharmaceutical formulation comprising cannabinoids, or a salt or derivative thereof, formulated in a nanoemulsion comprising droplets having an average particle size of less than about 1 micron, wherein the cannabinoids are present in the formulation at a concentration of 0.01% to 75.0% (w/w) of the formulation, and wherein the formulation further comprises a pharmaceutically acceptable oil having an oxidizable metal of less than one part per million.

16. A pharmaceutical formulation according to claim 15, wherein the formulation further comprises an anti-inflammatory, and the anti-inflammatory is one or more of prednisone and diclofenac sodium, or a salt or derivative thereof.

17. A pharmaceutical formulation according to claim 15, wherein the formulation further comprises methotrexate.

18. A pharmaceutical formulation according to claim 16, wherein the formulation further comprises methotrexate.

19. A method of treating arthritis conditions in a subject suffering joint pain, comprising topically administering a pharmaceutical formulation of claim 1 on the skin of a subject requiring such treatment.

20. The method of claim 19, wherein the pharmaceutical formulation further comprises an anti-inflammatory, and said anti-inflammatory is one or more of prednisone and diclofenac sodium, or a salt or derivative thereof.
